(19) United States
(12) Patent Application Publication
(10) Pub. No.: US 2025/0325589 A1
(43) Pub. Date: Oct. 23, 2025

(54) GLYCOPEPTIDE MEDIATED T CELL IMMUNITY AND ANTI-TUMOR EFFICACY IN CANCER

(71) Applicant: Gnubiotics Sciences SA, Epalinges (CH)

(72) Inventor: Adeyemi Adesokan, Epalinges (CH)

(73) Assignee: Gnubiotics Sciences SA, Epalinges (CH)

(21) Appl. No.: 19/183,744

(22) Filed: Apr. 18, 2025

Related U.S. Application Data
(60) Provisional application No. 63/636,674, filed on Apr. 19, 2024.

(51) Int. Cl. A61K 35/17; A61K 38/17; A61K 39/00; A61K 40/11; A61P 35/00; C07K 16/28; C12N 5/0783

(52) U.S. Cl. CPC A61K 35/17; A61K 38/1735; A61K 40/11; A61P 35/00; C07K 16/2818; C07K 16/2827; C12N 5/0636; A61K 2039/505

(57) ABSTRACT

Provided herein are methods for treating cancer by administering to a recipient mammal reprogrammed T cells. The reprogrammed T cells of the disclosure is produced by a method comprising administering to the donor mammal an effective amount of a composition comprising a plurality of glycopeptides as described herein. Also provided are methods of producing reprogrammed T cells. Also provided are methods for modulating dendritic cell or macrophage activity in a subject. The modulated dendritic cells or macrophages produced by such methods have improved antigen presentation and/or T cell priming activities.

### CROSS-REFERENCE TO RELATED PATENT APPLICATION

This application claims the benefit under 35 U.S.C. § 119 (e) of U.S. Provisional Application No. 63/636,674, filed Apr. 19, 2024, the entire contents of which are incorporated herein by reference.

### BACKGROUND

Despite great advances in anti-cancer drug development, only a fraction of cancer patients respond well to current state-of-the-art treatments. Recent studies showed that cancer cells exhibit altered glycosylation patterns on their surfaces, which significantly promote cancer progression and immune evasion (Reily et al., 2019). This “glyco-code” on tumor cells modulates innate and adaptive immune responses (Rodríguez et al., 2018) via interaction with lectin receptors on immune cells, suggesting that glycans could serve as a novel therapeutic target to promote anti-tumor immunity (Gray et al., 2020). The low immunogenicity of glycans and the induction of largely T cell-independent immune responses, however, renders the use of single glycans for anti-tumor immunization challenging. Here, we demonstrate that a mixture of multiple glycopeptide conjugates, which mimics the glycosylation pattern of malignant cells, promotes activation of anti-tumor T cells, enhances anti-tumor immune responses and is effective as a single agent or in combination with a PD-1 checkpoint inhibitor as an emerging novel treatment option for epithelial cancers.

Colorectal cancer (CRC) is the third most frequent cancer in adults and the second leading cause of cancer-related deaths (Siegel et al., 2023). Out of all CRC tumors, 95% are resistant to immune checkpoint inhibition (ICI)-therapy (Chalabi et al., 2020) and a multitude of treatment modalities including radiation, chemotherapy, biologicals, and tyrosine kinase inhibitors show limited success in advanced disease (Gandini et al., 2023), demonstrating the need for novel therapeutic approaches (Lin et al., 2023).

About 95% of all surface proteins are glycosylated thus through this post-translational modification and resulting altered glycosylation pattern able to influence immune recognition (Varki et al., 2022). The diversity of glycans is determined by the nature of their carbohydrate building blocks, how these are linked, their branching pattern, and the non-carbohydrate binding carrier (proteins or lipids) (Reily et al., 2019). In glycoproteins/peptides, glycans are linked to the hydroxyl group of Serine or Threonin (O-glycans) or to the nitrogen atom of Aspargine (N-glycans) (Reily et al., 2019). Glycans are highly tissue-, cell-, and protein-specific and altered glycosylation is a hallmark of epithelial tumors comprising the truncation of mucin-like O-glycan structures (e.g. the Thomsen-nouveau (Tn) antigen which consists of a single N-acetylgalactosamine (GalNAc), and the T antigen where a galactose (Gal) is added to the GalNAc residue of Tn) (Pinho & Reis, 2015). In epithelial tumors, T and Tn antigens are often extended through excessive sialylation (sTn and sT, respectively) (Pinho & Reis, 2015). These alterations in the glycosylation pattern can promote immune evasion and are associated with negative clinical outcome (Reily et al., 2019; Sosa Cuevas et al., 2023). Glycan epitopes on tumors interact with lectin receptors on immune cells (Rodríguez et al., 2018) and might be utilized as novel therapeutic targets (Gray et al., 2020). The low immunogenicity of sole glycans and the induction of largely T-cell-independent immune responses, however, make the use of single glycopeptide epitopes for classical immunization challenging (Gabba et al., 2023; Lodewijk et al., 2023; Mayordomo et al., 2004; Rosenbaum et al., 2020).

### SUMMARY OF THE DISCLOSURE

Another aspect of the disclosure is directed to a method for treating cancer in a mammal in need thereof comprising, or alternatively consisting essentially of, or yet further consisting of administering to the mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, thereby treating the cancer in the recipient mammal.

In some embodiments, a standard therapy for the cancer is 5FU.

In some embodiments, the cancer is selected from melanoma, breast cancer, lung cancer, or colorectal cancer (CRC).

In some embodiments, the cancer is Microsatellite stable (MSS) colorectal cancer.

In some embodiments, the composition is administered to the mammal for 28 days or more.

In some embodiments, the plurality of glycopeptides is derived from porcine gastrointestinal mucins, and the composition is obtained without subjecting the porcine gastrointestinal mucins to conditions or reagents that cause release of oligosaccharides from glycopeptides.

In some embodiments, the cancer is stage 3 or 4 cancer. In some embodiments, the cancer is resistant to checkpoint inhibitor therapy.

In some embodiments, the method further comprises, or alternatively consists essentially of, or yet further consists of administering to the recipient mammal an immune checkpoint inhibitor before, after, or simultaneously with the composition. In some embodiments, the immune checkpoint inhibitor is an anti-PD1 antibody or an anti-PDL1 antibody.

Another aspect of the disclosure is directed to a method for preparing reprogrammed T cells comprising, or alternatively consisting essentially of, or yet further consisting of administering to a donor mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, and isolating the reprogrammed T cells from the donor mammal, wherein the reprogrammed T cells are capable of targeting tumor cells in a recipient mammal.

In some embodiments, the method further comprises, or alternatively consists essentially of, or yet further consists of cryopreserving the isolated T cells.

In some embodiments, the composition is administered to the donor mammal for 28 days or more before isolating the T cells.

In some embodiments, the glycopeptides were derived from porcine gastrointestinal mucins, and the composition is obtained without subjecting the porcine gastrointestinal mucins or the partially purified fraction thereof to conditions or reagents that cause complete release of oligosaccharides from glycopeptides.

Another aspect of the disclosure is directed to an ex vivo or in vitro method for preparing reprogrammed T cells comprising, or alternatively consisting essentially of, or yet further consisting of contacting T cells from a mammal with an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, and isolating the reprogrammed T cells, wherein the reprogrammed T cells are capable of targeting tumor cells in a recipient mammal.

In some embodiments, the reprogrammed T cells are used for adoptive cell transfer.

Another aspect of the disclosure is directed to a method for modulating dendritic cell or macrophage activity comprising, or alternatively consisting essentially of, or yet further consisting of administering to a mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, thereby modulating dendritic cell or macrophage activity in the mammal. In some embodiments, the modulated dendritic cell or macrophage; wherein the modulated dendritic cell or macrophage has improved antigen presentation and/or T cell priming activities.

In some embodiments, the method further comprises, or alternatively consists essentially of, or yet further consists of administering to the mammal an immune checkpoint inhibitor before, after, or simultaneously with the composition.

In some embodiments, the immune checkpoint inhibitor is an anti-PD1 antibody or an anti-PDL1 antibody.

### BRIEF DESCRIPTION OF THE DRAWINGS

FIGS. 1A-1D: A novel formulation of multiple glycopeptides mimics the tumor glyco-code. A formulation of glycopeptides termed “GNU101” rich in O-glycans yielded a light brown powder. A) Graphical representation of our compound (left) and glycans found on tumor cells (left). B) The formulation was hydrolyzed and monosaccharides identified and quantified using HPAEC-PAD or C) subjected to beta-elimination and the released glycan profile analyzed via LC-MS/MS. D) Table showing relevant glycan structures in the product and their respective reported innate immune cell receptors.

FIGS. 2A-2E: Supplementation of GNU101 reduces tumor growth. 9-12-week-old C57BL/6 mice were injected on each flank (B, C, E) or into the cecum wall (D) with 300,000 MC38 cells and treated with 3% GNU101 in the drinking water starting two weeks before (prophylactic, B) or 6 days after (therapeutic approach, C-E) tumor cell injection. Starting on day 6 post tumor cell injection, the mice were injected every 3 days with an isotype control or an anti-PD-1 antibody (B-D), or with vehicle or 5-FU (E), as indicated. A) Schematic overview of the experimental layout. Tumor growth, representative pictures and tumor weight on day 15 after tumor cell injection from B) the prophylactic approach where MC38 cells were injected subcutaneously; and C) the therapeutic approach in MC38 cells injected subcutaneously. D) Tumor volume, representative pictures and tumor weight at the last day of the experiment in the therapeutic approach in MC38 cells injected into the cecum wall. E) Tumor growth, representative pictures and tumor weight on day 15 after tumor cell injection in the therapeutic GNU101 administration scheme in combination with 5-FU treatment. *p<0.05, **p<0.01, ***p<0.001. For graphs showing tumor growth over time n=8-10, data represent mean values of each group with error bars representing SEM. In bar graphs, each dot represents one tumor.

FIGS. 3A-3J: GNU101 administration promotes T cell activation in tumors. A-G: 9-12-week-old C57BL/6 mice were injected on each flank with 300,000 MC38 cells and treated with 3% GNU101 in the drinking water starting 6 days after tumor cell injection. On day 6, day 9 and day 12 post tumor cell injection, the mice were injected with an isotype control or an anti-PD-1 antibody. 15 days after tumor cell injection, tumors were harvested and the tissue analyzed for A) cytokine levels using a multiplex cytokine assay for the indicated cytokines; relative abundance of B) % macrophages among myeloid cells, C) % of IL-12+ cells among all macrophages, D) % of CD3+ cells among CD45+ cells, E) % of CD8+ cells among all CD3 cells using flow cytometry; F) abundance of CD8+ cells using IHC; and G) for % of Perforin+/granzymeB+ and IFN□+ cells among CD8+ T cells using flow cytometry. H&I: Tumors were harvested and single cell suspensions either left untreated or re-stimulated with GNU101 or PMA+Inonomycin for 6 h. H) Schematic overview of the experimental set up; I) Flow cytometry to determine the % of CD69+ and J) IFNγ+ cells among CD8+ T cells. In bar graphs, each dot represents analysis from one tumor. *p<0.05, **p<0.01, ***p<0.001.

FIGS. 4A-4D: Adoptive transfer of T cells from GNU-treated mice prevents tumor growth in treatment-naïve hosts. 9-12-week-old C57BL/6 mice were injected on each flank with 300,000 MC38 cells and treated with 3% GNU101 in the drinking water starting 6 days after tumor cell injection. On day 6, day 9 and day 12 post tumor cell injection, the mice were injected with an isotype control or an anti-PD-1 antibody. 15 days after tumor cell injection, tumors were harvested, and T cells isolated from the tumor tissue. The T cells were expanded for one week and injected i.p. into C547BL/6 recipient mice that were injected on each flank with 300,000 MC38 cells on the same day. Mice that received T cells from the spleen of non-tumor-bearing mice and mice that did not receive T cells served as controls. A) Schematic overview of the experimental layout. B) Tumor growth over time in donor mice (n=20), C) representative pictures, and D) weight of tumors isolated from T cell recipient mice on day 15 after tumor cell injection (n=10 per group). Data is presented as mean values of each group with error bars representing SEM.

FIGS. 5A-5G: GNU101 administration at 3% is most effective to reducing tumor growth and does not induce signs of liver or kidney toxicity. 9-12-week-old C57BL/6 mice were injected on each flank with 300,000 MC38 cells and treated with 1% or 3% GNU101 in the drinking water starting on day 6 after tumor cell injection. On day 6, day 9 and day 12 post-tumor cell injection, the mice were injected with an isotype control or an anti-PD-1 antibody. A) Schematic overview of the experimental layout. B) Tumor growth, C) representative pictures and D) tumor weight of tumors isolated on day 14 after tumor cell injection. Serum levels of E) aspartate aminotransferase (AST), F) alanine aminotransferase (ALT), and G) Creatinine at the end of the experiment. *=p<0.05, **=p<0.01. n=10, B: data represent mean values of each group with error bars representing SEM. D-G: each dot represents one tumor.

FIGS. 6A-6G: GNU101 administration reduces tumor growth in a melanoma and a breast cancer model. 9-12-week-old C57BL/6 mice were injected on each flank with 300,000 YUMM1.7 melanoma cells (B-D) or Balb/c mice with 500,000 4T1 breast carcinoma cells (E-G) and treated with 3% GNU101 in the drinking water starting two weeks before (YUMM1.7, prophylactic, B-D) or 6 days after (4T1, therapeutic approach, E-G) tumor cell injection. On day 6, day 9 and day 12 post-tumor cell injection, the mice were injected with an isotype control or an anti-PD-1 antibody. A) Schematic overview of the experimental layout. B&E) Tumor growth, C&F) representative pictures and D&G) tumor weight of tumors isolated on day 15 after tumor cell injection. *p<0.05, n=8 in the GNU101+aPD1 group, n=10 for all other groups. B&F: data represent mean values of each group with error bars representing SEM. D&G: each dot represents one tumor.

FIGS. 7A-7H: Supplementation of GNU101 promotes IFNγ+CD4+ T cells while suppressing Th17 and Treg cells in the tumor. 9-12-week-old C57BL/6 mice were injected on each flank with 300,000 MC38 cells and treated with 3% GNU101 in the drinking water starting 6 days after tumor cell injection. On day 6, day 9 and day 12 post-tumor cell injection, the mice were injected with an isotype control or an anti-PD-1 antibody. On day 15, the tumors were harvested and analyzed for the indicated cell populations using flow cytometry. A) B cells, B) Dentritic cells, C) Granulocytes, D) CD4− (of CD3+), E) Ifng+, F) TNFa+, G) IL-17+, or H) FcxP3 cells. ***p<0.001, n=8 in the aPD1 group, n=10 for all other groups. Each dot represents data from one tumor.

FIGS. 8A-8H: GNU101 administration promotes T cell activation in the prophylactic approach. 9-12-week-old C57BL/6 mice were injected on each flank with 300,000 MC38 cells (A-D) or YUMM1.7 melanoma cells (E-H) and treated with 3% GNU101 in the drinking water starting two weeks before tumor cell injection. On day 6, day 9 and day 12 post tumor cell injection, the mice were injected with an isotype control or an anti-PD-1 antibody. 15 days after tumor cell injection, tumors were harvested and analyzed for the indicated cell populations using flow cytometry. *p<0.05, n=10 for all groups, each dot represents data from one tumor.

FIGS. 9A-9C: GNU101 administration promotes apoptosis in the tumor tissue but has no effect on cell proliferation. 9-12-week-old C57BL/6 mice were injected on each flank with 300,000 MC38 cells and treated with 3% GNU101 in the drinking water starting 6 days after tumor cell injection. On day 6, day 9 and day 12 post tumor cell injection, the mice were injected with an isotype control or an anti-PD-1 antibody. (A) On day 15, the tumors were harvested and stained with hematoxylin and Eosin (H&E), with an antibody against cleaved caspase 3 (cl. casp3), or an antibody against Ki67. (B)-(C) The graphs show the number of positive cells per high-magnification field. *p<0.05, n=10 for all groups, each dot represents data from one tumor.

### DETAILED DESCRIPTION

### Definitions

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

An “effective amount” is an amount necessary to achieve the desired therapeutic or diagnostic result and will vary with the disease and subject or patient to be treated. Effective amounts are determined using methods known in the art and as described herein.

The expression “gastrointestinal tract mucins” encompasses any natural source of mucin from which glycans and glycopeptides can be extracted, suitable for mammalian nutrition or pharmaceutical use. Typical sources of gastrointestinal tract mucins are extracts from gastrointestinal tract, in particular from porcine sources or from bovine sources. Commercial sources for gastrointestinal tract mucins include Biofac A/S (Kastrup, Denmark), Zhongshi Duqing (Heze, China), Shenzhen Taier Biotechnology Co., LTD (Shenzhen, China), and Dongying Tiandong Pharmaceutical Co. (Shandong, China). In some embodiments, the gastrointestinal tract mucins are from porcine gastric mucus.

The expression “subject” refers to mammals. For examples, mammals contemplated by the present invention include human, primates, domesticated animals such as cattle, sheep, pigs, horses, rodents, cats, dogs, and other pets. In some embodiments, the subject is a human. In some embodiments, the subject may be an infant (1 year old or less for a human), a toddler (3 years old or less for a human), a child, a young adult, an adult or a geriatric. In some embodiments, the infant is a newborn. The subject may be male or female. In some embodiments, the subject is female and of child-bearing age.

The term “glycoprotein” refers to proteins linked to oligosaccharides, e.g., proteins either N-linked or O-linked to oligosaccharides, and having a molecular weight of more than about 5 KDa.

The term “glycopeptide” refers to peptides linked to oligosaccharides, e.g. peptides either N-linked or O-linked to oligosaccharides, and having a molecular weight of less than about 5 KDa. Methods of determining molecular weight of glycopeptides and glycoproteins are known in the art and are not limited. In some embodiments, the molecular weight of glycopeptides and glycoproteins are determined by size exclusion chromatography.

The term “glycan” as used herein refers to an oligosaccharide. The term “free glycan” is synonymous with the term “free oligosaccharide,” as also used herein.

In some embodiments, peptides are defined as having a molecular weight of less than about 5 KDa. In some embodiments, the term peptides include glycopeptides. In some embodiments, proteins are defined as having a molecular weight of more than about 5 KDa. In some embodiments, the term proteins include glycoproteins.

As used herein, “a partially purified fraction” of gastrointestinal tract mucins comprises, or alternatively consists essentially of, or yet further consists of at least about 40%, at least about 50%, at least about 60%, at least about 70%, at least about 80%, at least about 90%, at least about 92.5%, at least about 95%, at least about 97.5%, at least about 98%, at least about 99%, or at least about 99.5% of the protein- and peptide-glycans present in un-purified gastrointestinal tract mucins. In some embodiments, the mucins or partially purified fraction thereof has been subject to an acid treatment.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. In another aspect, the term “treatment” excludes prophylaxis. Alternatively, treatment is “effective” if the progression of a disease is reduced or halted. “Treatment” can also mean prolonging survival as compared to expected survival if not receiving treatment.

The term “chemotherapy” refers to administration of any genotoxic agent (e.g., DNA damaging agent), including conventional or non-conventional chemotherapeutic agents, for the treatment or prevention of cancer. Chemotherapeutic agents include agents that have been modified, (e.g., fused to antibodies or other targeting agents). Examples of chemotherapeutic agents include, but are not limited to, platinum compounds (e.g, cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine, mitomycin C), antitumor antibiotics (e.g., daunorubicin, doxorubicin, idarubicin, epirubicin, mitoxantrone, bleomycin, plicamycin, dactinomycin), taxanes (e.g., paclitaxel, /raZ>-paclitaxel and docetaxel), antimetabolites (e.g: 5-fluorouracil, cytarabine, premetrexed, thioguanine, floxuridine, capecitabine, and methotrexate), nucleoside analogues (e.g., fludarabine, clofarabine, cladribine, pentostatin, nelarabine, gemcitabine, 5-flurouracil), topoisomerase inhibitors (e.g., topotecan, irinotecan, SN-38, CPT-11), hypomethylating agents (e.g., azacitidine and decitabine), proteasome inhibitors (e.g., bortezomib), epipodophyllotoxins (e.g., etoposide and teniposide), DNA synthesis inhibitors (e.g., hydroxyurea), and vinca alkaloids (e.g., vincristine, vindesine, vinorelbine, and vinblastine). Chemotherapeutic agents include DNA intercalating agents (e.g, pyrrol obenzodiazepines).

The terms, “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

Several classes of checkpoint inhibitors/regulators (immune checkpoint inhibitors “ICI”) are known in the art, including lymphocyte activation gene-3 (LAG-3), T cell immunoglobulin and ITIM domain (TIGIT), T cell immunoglobulin and mucin-domain containing-3 (TIM-3), V-domain immunoglobulin suppressor of T cell activation (VISTA), B7 homolog 3 protein (B7-H3), inducible T cell costimulatory (ICOS) and B and T lymphocyte attenuator (BTLA), anti-cytotoxic T lymphocyte associated antigen-4 (CTLA-4) as well as inhibitors of CTLA-4, programmed death 1 (PD-1; also referred to herein as PD1), and programmed death ligand-1 (PD-L1).

The latter 3 classes of checkpoint inhibitors, CTLA-4, PD-1 and PD-L1 inhibitors, have contributed several medically relevant drugs such as monoclonal antibody (mAb) inhibitors. Example of anti-CTLA-4 inhibitory mAb is ipilimumab (approved globally). Example of anti-PD-L1 inhibitory mAbs are atezolizumab, avelumab and durvalumab (approved globally). Non-limiting examples of anti-PD1 inhibitor mAbs include pembrolizumab and nivolumab (approved globally); sintilimab, tislelizumab, toripalimab, and camrelizumab (approved in China); geptanolimab serplulimab zimberelimab cemiplimab, dostarlimab, prolgolimab, balstilimab, penpulimab, retifanlimab, cadonilimab, pucotenlimab, sasanlimab, and cetrelimab.

Checkpoint inhibitory mAbs of distinct subclasses can be combined in a distinct modality (e.g. ipilimumab+nivolumab), or combined individually with chemotherapy, biologic therapies, anti-angiogenic therapies such as VEGF inhibitors, anti-TGFβs, cell therapies, mRNA therapies and so on.

### Modes for Carrying Out the Disclosure

The present disclosure investigated whether a formulation of multiple poly-O-glycopeptide conjugates that mimic the glycosylation pattern on malignant cells, can augment anti-tumor T cell immune responses. The present disclosure showed that this novel class of complex glycopolypeptides promotes anti-tumor immunity in a stand-alone approach and augments immune activation mediated by anti-PD1 immune checkpoint inhibition.

The present methods are based, at least in part, on the surprising ability to program T cells via glycan-mediated mechanisms. Specifically, the present methods allow target tumor types that evade the adaptive immune response through lectin-glycan interactions (e,g., MSS colorectal cancer) and allow the body's T cells to kill the tumors and their metastases wherever they are. By intervening at this glycan interface, Applicant demonstrates the capacity to re-educate T cells to recognize and target these tumors, which are otherwise non-responsive to standard immunotherapies. The present disclosure includes use of defined glycopeptide structures or glycan motifs to reprogram or train naive or memory T cells to recognize “invisible” tumors. In addition, the disclosed compositions that comprise glycan-based adjuvants enable this reprogramming in vivo or ex vivo.

The present disclosure also includes use of glycan-based immune training in combination with: Immune checkpoint inhibitors (e.g., anti-PD-1 or anti-PD-L1 antibodies), CAR-T or TCR therapies, and/or cancer vaccines. Synergistic benefit is shown in non-responsive (cold) tumors which normally evade immune response (e.g., MSS CRC).

The preparation of GNU101 (also referred to as GCX) is described in Applicant's prior published patent application WO2023/041979, which is incorporated herein in its entirety.

Structures are given in the text according following rules: the structure is described clockwise and left-to-right wherein the rightmost saccharide locates the attachment point to the peptide portion of the glycopeptide. Abbreviations used in glycan structures are as follows: Fuc, fucose; Gal, galactose; GalNAc, N-acetyl-galactose, Glc, glucose; GlcNAc, N-acetyl-glucosamine; NeuAc, N-acetyl-neuraminic acid; NeuGc, N-acetyl glycolylneuraminic acid; S, sulfate.

### Methods for Treating Cancer

In one embodiment, the disclosure provides an improved method of treating a tumor in a subject, particularly a human subject, wherein the standard of care for treating the tumor is administration of 5-flurouracil (5-FU), the improvement comprising administering to the subject in place of 5-FU a composition comprising a mixture of glycopeptides derived from porcine gastrointestinal tract mucins, each having a molecular weight of less than or equal to 5 kilodaltons, wherein the composition comprises less than about 25% (w/w) of free glycans (i.e., glycans that are not part of a glycopeptide) (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or less) and less than about 12% (w/w) (e.g., 12%, 11%, 10%, 9%, 8%, 7%, 6%, 5%, 4%, 3%, 2%, 1%, or lower) of proteins having a molecular weight of more than 5 kilodaltons, and wherein the mixture of glycopeptides comprises at least 5 (e.g., 5, 6, 7, 8, 9, 10, 11, 12, 13, 14, 15, or all 16) of the following glycan structures:
Fucα1-2Galβ1-3GalNAc
Galβ1-3(GlcNAcβ1-6)GalNAc1
Galβ1-3(6SGlcNAcβ1-6)GalNAc
Galβ1-3(NeuAcα2-6)GalNAc2
NeuAcα2-3Galβ1-3GalNAc3
Galβ1-3(NeuGcα2-6)GalNAc
NeuGcα2-3Galβ1-3GalNAc
GlcNAcβ1-3(6S-GlcNAcβ1-6)GalNAc
Galβ1-3(Galβ1-4GlcNAcβ1-6)GalNAc
Fucα1-2Galβ1-3(6S-GlcNAcβ1-6)GalNAc
Galβ1-3(Galα1-3Galβ1-4GlcNAcβ1-6)GalNAc
NeuAcα2-3Galβ1-3 [(6S)GlcNAcβ1-6]GalNAc
NeuAcα2-3Galβ1-3(NeuAcα2-6)GalNAc
NeuGcα2-3Galβ1-3(NeuAcα2-6)GalNAc
NeuGcα2-3Galβ1-3(NeuGcα2-6)GalNAc
GalNAcα1-3 (Fucα1-2)Galβ1-3(6SGlcNAcβ1-6)GalNAc.

In some aspects of this embodiment, the composition is GNU101. In some aspects of this embodiment, the tumor is a colorectal cancer. In some aspects of this embodiment, the tumor is a microsatellite instability (MSI) colorectal cancer. In some aspects of this embodiment, the tumor is a microsatellite instability-high (MSI-H) colorectal cancer. In some aspects of this embodiment, the tumor is a microsatellite instability-low (MSI-L) colorectal cancer. In some aspects of this embodiment, the tumor is a microsatellite stable (MSS) colorectal cancer. In some aspects of this embodiment, the tumor is a melanoma. In some aspects of this embodiment, the tumor is a breast cancer.

5-FU is a chemotherapeutic agent that is used in the treatment of various cancers, including anal, breast, colorectal, oesophageal, stomach, pancreatic, cervical and skin cancers (especially head and neck cancers). It is the standard of care in MSS colorectal cancer and has often been used in the treatment of MSI colorectal cancer. It is an agent that prevents actively dividing cells from making and repairing DNA. 5-FU causes many side effects that can be debilitating for the subject. We believe that GNU101, with its effect on T-cell activation and tumor-specific T-cell targeting, provides a less toxic, but equally effective way to treat tumors and can therefore be used to replace 5-FU in tumor treatment.

In some aspects of these embodiments, the subject is also treated with an immunooncology agent. In some aspects, the immunooncology agent is or comprises an anti-PD1 antibody.

An aspect of the disclosure is directed to a method for treating cancer in a recipient mammal in need thereof comprising, or alternatively consisting essentially of, or yet further consisting of administering to a mammal an effective amount of T cells isolated from a donor mammal. The T cells were prepared by a method comprising, or alternatively consisting essentially of, or yet further consisting of administering to the donor mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, thereby treating the cancer in the recipient mammal.

In some embodiments, the cancer is selected from melanoma, breast cancer, lung cancer, or colorectal cancer (CRC).

In some embodiments, the cancer is Microsatellite stable (MSS) colorectal cancer.

In some embodiments, the T cells are autologous or allogeneic to the recipient mammal, or the T cells are HLA1-deficient T cells or TCR-deficient T cells, or the composition is administered to the donor mammal for 28 days or more before isolating the T cells.

In some embodiments, the plurality of glycopeptides is derived from porcine gastrointestinal mucins, and the composition is obtained without subjecting the porcine gastrointestinal mucins to conditions or reagents that cause release of oligosaccharides from glycopeptides.

In some embodiments, the cancer is stage 3 or 4 cancer. In some embodiments, the cancer is resistant to checkpoint inhibitor therapy.

In some embodiments, the method further comprises, or alternatively consists essentially of, or yet further consists of administering to the recipient mammal an immune checkpoint inhibitor before, after, or simultaneously with the isolated T cells. In some embodiments, the immune checkpoint inhibitor is an anti-PD1 antibody or an anti-PDL1 antibody.

Another aspect of the disclosure is directed to a method for preparing reprogrammed T cells comprising, or alternatively consisting essentially of, or yet further consisting of administering to a donor mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, and isolating the reprogrammed T cells from the donor mammal, wherein the reprogrammed T cells are capable of targeting tumor cells in a recipient mammal.

In some embodiments, the method further comprises, or alternatively consists essentially of, or yet further consists of cryopreserving the isolated T cells.

In some embodiments, the composition is administered to the donor mammal for 28 days or more before isolating the T cells.

In some embodiments, the glycopeptides were derived from porcine gastrointestinal mucins, and the composition is obtained without subjecting the porcine gastrointestinal mucins or the partially purified fraction thereof to conditions or reagents that cause complete release of oligosaccharides from glycopeptides.

Another aspect of the disclosure is directed to a method for treating cancer in a mammal in need thereof comprising, or alternatively consisting essentially of, or yet further consisting of administering to the mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, thereby treating the cancer in the recipient mammal.

In some embodiments, a standard therapy for the cancer is 5FU.

In some embodiments, the cancer is selected from melanoma, breast cancer, lung cancer, or colorectal cancer (CRC).

In some embodiments, the cancer is Microsatellite stable (MSS) colorectal cancer.

In some embodiments, the composition is administered to the mammal for 28 days or more.

In some embodiments, the plurality of glycopeptides is derived from porcine gastrointestinal mucins, and the composition is obtained without subjecting the porcine gastrointestinal mucins to conditions or reagents that cause release of oligosaccharides from glycopeptides.

In some embodiments, the cancer is stage 3 or 4 cancer. In some embodiments, the cancer is resistant to checkpoint inhibitor therapy.

In some embodiments, the method further comprises, or alternatively consists essentially of, or yet further consists of administering to the recipient mammal an immune checkpoint inhibitor before, after, or simultaneously with the composition. In some embodiments, the immune checkpoint inhibitor is an anti-PD1 antibody or an anti-PDL1 antibody.

In some embodiments, the composition disrupts lectin-glycan interactions involved in immune evasion.

In some embodiments, the composition converts an immune-desert tumor (a tumor that has evaded immune response and is therefore substantially immune cell free) microenvironment into an inflamed tumor microenvironment which attracts immune cells and immune response, thereby facilitating tumor cell destruction.

Methods for Programming T cells

In one embodiment, the disclosure provides a method of programming a subject's T-cells to target a tumor in a subject, particularly a human subject, comprising the step of administering to the subject a pharmaceutically effective amount of a composition comprising a mixture of glycopeptides derived from porcine gastrointestinal tract mucins, each having a molecular weight of less than or equal to 5 kilodaltons, wherein the composition comprises less than about 25% (w/w) of free glycans (i.e., glycans that are not part of a glycopeptide) (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or less) and less than about 12% (w/w) (e.g., 12%, 11%, 10%, 9%, 8%, 7%, 6%, 5%, 4%, 3%, 2%, 1%, or lower) of proteins having a molecular weight of more than 5 kilodaltons, and wherein the mixture of glycopeptides comprises at least 5 (e.g., 5, 6, 7, 8, 9, 10, 11, 12, 13, 14, 15, or all 16) of the following glycan structures:
Fucα1-2Galβ1-3GalNAc
Galβ1-3(GlcNAcβ1-6)GalNAc1
Galβ1-3(6SGlcNAcβ1-6)GalNAc
Galβ1-3(NeuAcα2-6)GalNAc2
NeuAcα2-3Galβ1-3GalNAc3
Galβ1-3(NeuGcα2-6)GalNAc
NeuGcα2-3Galβ1-3GalNAc
GlcNAcβ1-3(6S-GlcNAcβ1-6)GalNAc
Galβ1-3(Galβ1-4GlcNAcβ1-6)GalNAc
Fucα1-2Galβ1-3(6S-GlcNAcβ1-6)GalNAc
Galβ1-3(Galα1-3Galβ1-4GlcNAcβ1-6)GalNAc
NeuAcα2-3Galβ1-3 [(6S)GlcNAcβ1-6]GalNAc
NeuAcα2-3Galβ1-3(NeuAcα2-6)GalNAc
NeuGcα2-3Galβ1-3(NeuAcα2-6)GalNAc
NeuGcα2-3Galβ1-3(NeuGcα2-6)GalNAc
GalNAcα1-3 (Fucα1-2)Galβ1-3(6SGlcNAcβ1-6)GalNAc.

In some aspects of this embodiment, the composition is GNU101. In some aspects of this embodiment, the tumor is a colorectal cancer. In some aspects of this embodiment, the tumor is a microsatellite instability (MSI) colorectal cancer. In some aspects of this embodiment, the tumor is a microsatellite instability-high (MSI-H) colorectal cancer. In some aspects of this embodiment, the tumor is a microsatellite instability-low (MSI-L) colorectal cancer. In some aspects of this embodiment, the tumor is a microsatellite stable (MSS) colorectal cancer. In some aspects of this embodiment, the tumor is a melanoma. In some aspects of this embodiment, the tumor is a breast cancer. In some embodiments, the composition is capable of producing T-cells that are more specific and more active against any of the foregoing cancers than pre-existing T-cells that are not activated by such composition. The T-cells produced by administration of this composition is therefore less likely to recognize any self-antigens in the subject and therefore is likely to be less toxic than other T-cell activating therapies. In some embodiments, the T-cells produced by administration of the composition described above are resistant to PD-1-drive exhaustion. In some aspects of these embodiments, the subject is also treated with an immunooncology agent. In some aspects, the immunooncology agent is or comprises an anti-PD1 antibody.

Another aspect of the disclosure is directed to a method for preparing reprogrammed T cells comprising, or alternatively consisting essentially of, or yet further consisting of administering to a donor mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, and isolating the reprogrammed T cells from the donor mammal, wherein the reprogrammed T cells are capable of targeting tumor cells in a recipient mammal.

In some embodiments, the method further comprises, or alternatively consists essentially of, or yet further consists of cryopreserving the isolated T cells.

In some embodiments, the composition is administered to the donor mammal for 28 days or more before isolating the T cells.

In some embodiments, the glycopeptides were derived from porcine gastrointestinal mucins, and the composition is obtained without subjecting the porcine gastrointestinal mucins or the partially purified fraction thereof to conditions or reagents that cause complete release of oligosaccharides from glycopeptides.

Another aspect of the disclosure is directed to an ex vivo or in vitro method for preparing reprogrammed T cells comprising, or alternatively consisting essentially of, or yet further consisting of contacting T cells from a mammal with an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, and isolating the reprogrammed T cells, wherein the reprogrammed T cells are capable of targeting tumor cells in a recipient mammal.

Another aspect of the disclosure is directed to engineering T Cells to recognize glycan-encoded signals comprising, or alternatively consisting essentially of, or yet further consisting of contacting T cells from a mammal with an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, and isolating the reprogrammed T cells, wherein the reprogrammed T cells are capable of targeting tumor cells in a recipient mammal.

In some embodiments, the reprogrammed T cells are used for adoptive cell transfer.

### Methods for Activating T Cells Against a Tumor

In one embodiment, the disclosure provides a method of activating a subject's T-cells to target a tumor in a subject, particularly a human subject, comprising the step of administering to the subject a pharmaceutically effective amount of a composition comprising a mixture of glycopeptides derived from porcine gastrointestinal tract mucins, each having a molecular weight of less than or equal to 5 kilodaltons, wherein the composition comprises less than about 25% (w/w) of free glycans (i.e., glycans that are not part of a glycopeptide) (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or less) and less than about 12% (w/w) (e.g., 12%, 11%, 10%, 9%, 8%, 7%, 6%, 5%, 4%, 3%, 2%, 1%, or lower) of proteins having a molecular weight of more than 5 kilodaltons, and wherein the mixture of glycopeptides comprises at least 5 (e.g., 5, 6, 7, 8, 9, 10, 11, 12, 13, 14, 15, or all 16) of the following glycan structures:
Fucα1-2Galβ1-3GalNAc
Galβ1-3(GlcNAcβ1-6)GalNAc1
Galβ1-3(6SGlcNAcβ1-6)GalNAc
Galβ1-3(NeuAcα2-6)GalNAc2
NeuAcα2-3Galβ1-3GalNAc3
Galβ1-3(NeuGcα2-6)GalNAc
NeuGcα2-3Galβ1-3GalNAc
GlcNAcβ1-3(6S-GlcNAcβ1-6)GalNAc
Galβ1-3(Galβ1-4GlcNAcβ1-6)GalNAc
Fucα1-2Galβ1-3(6S-GlcNAcβ1-6)GalNAc
Galβ1-3(Galα1-3Galβ1-4GlcNAcβ1-6)GalNAc
NeuAcα2-3Galβ1-3 [(6S)GlcNAcβ1-6]GalNAc
NeuAcα2-3Galβ1-3(NeuAcα2-6)GalNAc
NeuGcα2-3Galβ1-3(NeuAcα2-6)GalNAc
NeuGcα2-3Galβ1-3(NeuGcα2-6)GalNAc
GalNAcα1-3 (Fucα1-2)Galβ1-3(6SGlcNAcβ1-6)GalNAc.

In some aspects of this embodiment, the composition is GNU101. In some aspects of this embodiment, the tumor is a colorectal cancer. In some aspects of this embodiment, the tumor is a microsatellite instability (MSI) colorectal cancer. In some aspects of this embodiment, the tumor is a microsatellite instability-high (MSI-H) colorectal cancer. In some aspects of this embodiment, the tumor is a microsatellite instability-low (MSI-L) colorectal cancer. In some aspects of this embodiment, the tumor is a microsatellite stable (MSS) colorectal cancer. In some aspects of this embodiment, the tumor is a melanoma. In some aspects of this embodiment, the tumor is a breast cancer. In some aspects of these embodiments, the subject is also treated with an immunoncology agent. In some aspects, the immunooncology agent is or comprises an anti-PD1 antibody.

### Methods for Modulating Dendritic Cell or Macrophage Activity

Another aspect of the disclosure is directed to a method for modulating dendritic cell or macrophage activity comprising, or alternatively consisting essentially of, or yet further consisting of administering to a mammal an effective amount of a composition comprising, or alternatively consisting essentially of, or yet further consisting of a plurality of glycopeptides, wherein at least 90% of the plurality of glycopeptides in the composition are less than 2 KDa in size, and wherein the plurality of glycopeptides comprises, or alternatively consists essentially of, or yet further consists of specific oligosaccharide structures, thereby modulating dendritic cell or macrophage activity in the mammal. In some embodiments, the modulated dendritic cell or macrophage; wherein the modulated dendritic cell or macrophage has improved antigen presentation and/or T cell priming activities.

In some embodiments, the method further comprises, or alternatively consists essentially of, or yet further consists of administering to the mammal an immune checkpoint inhibitor before, after, or simultaneously with the composition.

In some embodiments, the immune checkpoint inhibitor is an anti-PD1 antibody or an anti-PDL1 antibody.

The following examples are provided to illustrate, and not limit the scope of this disclosure.

### EXAMPLES

### Structure of GNU101

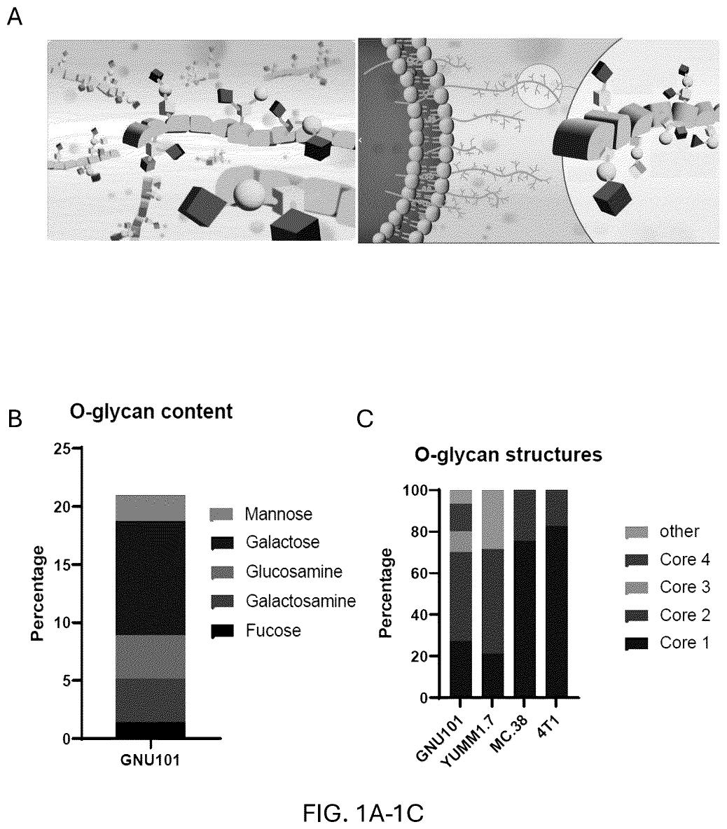

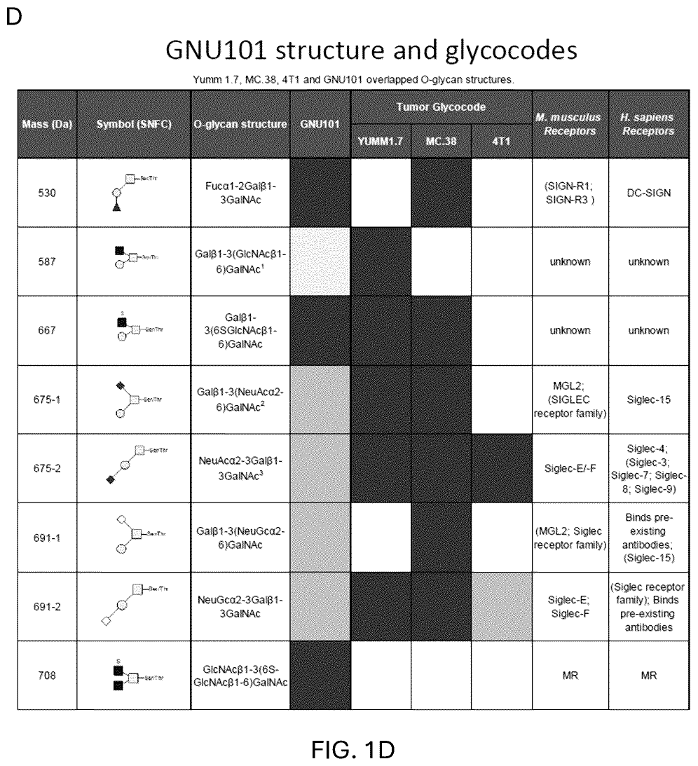

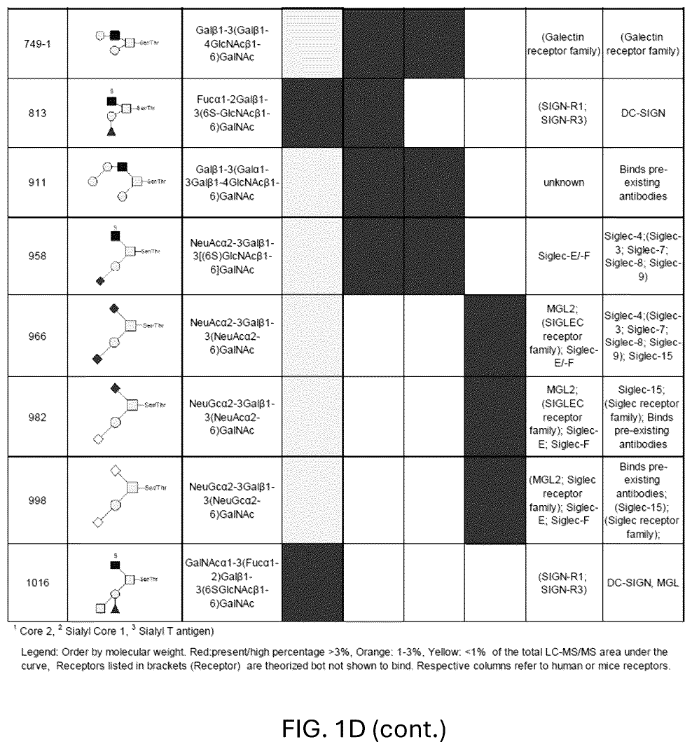

To obtain a mixture of glycans mimicking the glycosylation pattern found on tumor cells, a proprietary rational design process was used to obtain a defined mixture of O-glycosylated peptides comprising of 101 unique glycans termed GNU101. The glycopeptides had an average size of 2-5 kDa and contained over 21% w/w glycan content (FIG. 1A-1B). LC-MS/MS analysis (Karlsson et al., 2009) confirmed that these glycans included sT and sialylated core 1 canonical tumor glycans and 41% sialylated structures (FIG. 1D, List 1 below).

List 1: GNU101 Structures

| Composition | Putative structures |
|---|---|
| Hex1HexNAc2deHex1Sul1 | Fucα1-2Galβ1-3(6S-GlcNAcβ1-6)GalNAcol |
| Hex1HexNAc2Sul1 | Galβ1-3(6SGlcNAcβ1-6)GalNAcol |
| Hex1HexNAc1deHex1 | Fucα1-2Galβ1-3GalNAcol |
| Hex1HexNAc3deHex1Sul1 | GalNAcα1-3(Fucα1-2)Galβ1-3(6SGlcNAcβ1-6)GalNAcol |
| HexNAc3Sul1 | GlcNAcβ1-3(6S-GlcNAcβ1-6)GalNAcol |
| Hex1HexNAc2deHex1 | Fucα1-2Galβ1-3GlcNAcβ1-3GalNAcol |
| Hex1HexNAc2deHex1 | Fucα1-2(GalNAcα1-3)Galβ1-3GalNAcol |
| NeuAc1Hex1HexNAc1 | Galβ1-3(NeuAcα2-6)GalNAcol |
| NeuAc1Hex1HexNAc1deHex1 | Fucα1-2Galβ1-3(NeuAcα2-6)GalNAcol |
| Hex1HexNAc3deHex1Sul1 | Fucα1-2Galβ1-4GlcNAcβ-3(6SGlcNAcβ1-6)GalNAcol |
| NeuAc1Hex1HexNAc1 | NeuAcα2-3Galβ1-3GalNAcol |
| Hex1HexNAc3Sul1 | Galβ1-4GlcNAcβ1-3(6SGlcNAcβ1-6)GalNAcol |
| Hex1HexNAc1 | Galβ1-3GalNAcol |
| NeuAc1HexNAc2 | GalNAcα1-3(NeuAcα2-6)GalNAcol |
| NeuAc1HexNAc1 | NeuAcα2-6GalNAcol |
| NeuAc1HexNAc2 | GlcNAcβ1-3(NeuAcα2-6)GalNAcol |
| HexNAc2 | GalNAcα1-3GalNAcol |
| HexNAc2 | GlcNAcβ1-3GalNAcol |
| Hex2HexNAcSul1 | Galβ1-3[Galβ1-4(6S)GlcNAcβ1-6]GalNAcol |
| NeuGc1Hex1HexNAc1 | Galβ1-3(NeuGcα2-6)GalNAcol |
| NeuGc1Hex1HexNAc1 | NeuGcα2-3Galβ1-3GalNAcol |
| NeuGc1Hex1HexNAc2Sul1 | NeuGcα2-6Galβ1-3(6SGlcNAcβ1-6)GalNAcol |
| NeuAc1Hex1HexNAc2deHex1 | Fucα1-2Galβ1-4GlcNAcβ1-3(NeuAcα2-6)GalNAcol |
| Hex2HexNAc2deHex2Sul1 | Fucα1-2Galβ1-3[Fucα1-2Galβ1-4(6S)GlcNAcβ1-6]GalNAcol |
| Hex1HexNAc4deHex1Sul1 | GalNAcα1-3(Fucα1-2)Galβ1-3GlcNAcβ1-3(6SGlcNAcβ1-6)GalNAcol |
| NeuAc1Hex1HexNAc2 | GlcNAcα1-4Galβ1-3(NeuAcα2-6)GalNAcol |
| NeuAc1Hex1HexNAc2Sul1 | NeuAcα2-3Galβ1-3[(6S)GlcNAcβ1-6]GalNAcol |
| NeuAc1Hex1HexNAc2 | NeuAcα2-3(GalNAcβ1-4)Galβ1-3GalNAcol |
| Hex1HexNAc2 | Galβ1-4GlcNAcβ1-3GalNAcol |
| NeuAc1Hex1HexNAc3Sul1 | NeuAcα2-3(GalNAcβ1-4)Galβ1-3(6SGlcNAcβ1-6)GalNAcol |
| Hex1HexNAc3deHex1 | Fucα1-2(GalNAcα1-3)Galβ1-4GlcNAcβ1-3GalNAcol |
| NeuGc1HexNAc2 | GalNAcα1-3(NeuGcα2-6)GalNAcol |
| NeuGc1Hex1HexNAc1deHex1 | Fucα1-2Galβ1-3(NeuGcα2-6)GalNAcol |
| NeuGc1HexNAc2 | GlcNAcβ1-3(NeuGcα2-6)GalNAcol |
| Hex2HexNAc2deHex1Sul1 | Galβ1-3[Fucα1-2Galβ1-4(6S)GlcNAcβ1-6]GalNAcol |
| Hex1HexNAc3deHex1Sul1 | Fucα1-2Galβ1-4(6S)GlcNAcβ1-3(GlcNAcβ1-6)GalNAcol |
| Hex2HexNAc4deHex2Sul1 | GlcNAcα1-4(Fucα1-2)GlcNAcβ1-3[Fucα1-2)Galβ1-4(6S)GlcNAcβ1-6]GalNAcol |
| Hex1HexNAc3Sul1 | GlcNAcβ1-3[Galβ1-4(6S)GlcNAcβ1-6]GalNAcol |
| NeuGc1HexNAc1 | NeuGcα2-6GalNAcol |
| Hex1HexNAc2deHex1 | Fucα1-2Galβ1-3(GlcNAcβ1-6)GalNAcol |
| Hex2HexNAc2deHex2 | Fucα1-2Galβ1-3(Fucα1-2Galβ1-4GlcNAcβ1-6)GalNAcol |
| Hex2HexNAc2 | Galβ1-3(Galβ1-4GlcNAcβ1-6)GalNAcol |
| NeuGc1Hex1HexNAc2 | GalNAcβ1-4(NeuGcα2-3)Galβ1-3GalNAcol |
| NeuGc1Hex1HexNAc2deHex1 | Fucα1-2Galβ1-4GlcNAcβ1-3(NeuGcα2-6)GalNAcol |
| Hex2HexNAc2deHex1 | Galβ1-3(Fucα1-2Galβ1-4GlcNAcβ1-6)GalNAcol |
| Hex1HexNAc3Sul1 | GlcNAcα1-4Galβ1-3[(6S)GlcNAcβ1-6]GalNAcol |
| Hex2HexNAc2deHex1Sul1 | Fucα1-2Galβ1-3[Galβ1-4(6S)GlcNAcβ1-6]GalNAcol |
| Hex2HexNAc3deHex2Sul1 | Fucα1-2Galβ1-4GlcNAcβ1-3[Fucα1-2Galβ1-4(6S)GlcNAcβ1-6]GalNAcol |
| Hex1HexNAc2 | Galβ1-3(GlcNAcβ1-6)GalNAcol |
| Hex2HexNAc3deHex1Sul1 | Galβ1-3[GalNAcα1-3(Fucα1-2)Galβ1-4(6S)GlcNAcβ1-6]GalNAcol |
| Hex3HexNAc2 | Galα1-3Galβ1-4GlcNAcβ1-3Galβ1-3GalNAcol |
| Hex2HexNAc3Sul1 | Galβ1-4GlcNAcβ1-3[Galβ1-4(6S)GlcNAcβ1-6]GalNAcol |
| Hex1HexNAc3deHex1 | GlcNAcβ1-3(Fucα1-2Galβ1-4GlcNAcβ1-6)GalNAcol |
| NeuAc1Hex1HexNAc3deHex1 | GlcNAcα1-4(Fucα1-2)Galβ1-4GlcNAcβ1-3(NeuAcα2-6)GalNAcol |
| NeuGc1Hex1HexNAc3Sul1 | GlcNAcβ1-3[NeuGcα2-3Galβ1-4(6S)GlcNAcβ1-6]GalNAcol |
| Hex1HexNAc3 | GlcNAcβ1-3(Galβ1-4GlcNAcβ1-6)GalNAcol |
| Hex1HexNAc3deHex1Sul1 | GlcNAcβ1-3[Fucα1-2Galβ1-4(6S)GlcNAcβ1-6]GalNAcol |
| Hex2HexNAc3deHex2Sul1 | Fucα1-2Galβ1-3[GalNAcα1-3(Fucα1-2)Galβ1-3(6S)GlcNAcβ1-6]GalNAcol |
| NeuAc1Hex5HexNAc4deHex1 | NeuAcα2-Galβ1-4GlcNAcβ1-2Manα1-3(Galβ1-4GlcNAcβ1-2Manα1-6)Manβ1-4GlcNAcβ1-4(Fucα1- |
| | 6)GlcNAcol |
| Hex2HexNAc2deHex1 | Fucα1-2Galβ1-3(Galβ1-4GlcNAcβ1-6)GalNAcol |
| Hex1HexNAc1Sul | 3SGalβ1-3GalNAcol |
| HexNAc2Sul1 | 6SGlcNAcβ1-6GalNAcol |
| NeuAc2Hex1HexNAc1 | NeuAcα2-3Galβ1-3(NeuAcα2-6)GalNAcol |
| NeuGc1Hex2HexNAc2 | NeuGcα2-3Galβ1-3(Galβ1-4GlcNAcβ1-6)GalNAcol |
| NeuGc2Hex1HexNAc1 | NeuGcα2-3Galβ1-3(NeuGcα2-6)GalNAcol |
| Hex2HexNAc2 | Galβ1-3GlcNAcβ1-3Galβ1-3GalNAcol |
| NeuGc1Hex1HexNAc2 | NeuGcα2-3Galβ1-3(GlcNAcβ1-6)GalNAcol |
| HexNAc2Sul1 | 6SGlcNAcβ1-3GalNAcol |
| Hex2HexNAc3deHex2Sul1 | Fucα1-2Galβ1-3[GalNAcα1-3(Fucα1-2)Galβ1-4(6S)GlcNAcβ1-6]GalNAcol |
| Hex2HexNAc1deHex1 | Fucα1-2Galβ-4GlcNAcβ1-3Galol |
| Hex2HexNAc1 | Galβ1-4GlcNAcβ1-3Galol |
| HexNAc3 | GlcNAcβ1-3(GlcNAcβ1-6)GalNAcol |
| Hex3HexNAc2 | Galβ1-3(Galα1-3Galβ1-4GlcNAcβ1-6)GalNAcol |
| Hex2HexNAc5deHex2Sul1 | GalNAcα1-3(Fucα1-2)Galβ1-4GlcNAcβ1-3[GalNAcα1-3(Fucα1-2)Galβ1-4(6S)GlcNAcβ1-6]GalNAcol |
| Hex2HexNAc3deHex1Sul1 | Fucα1-2Galβ1-4GlcNAcβ1-3[Galβ1-4(6S)GlcNAcβ1-6]GalNAcol |
| Hex2HexNAc3deHex1Sul1 | Galβ1-4GlcNAcβ1-3[Fucα1-2Galβ1-3(6S)GlcNAcβ1-6]GalNAcol |
| NeuAc2Hex1HexNAc2 | NeuAcα2-3(GalNAcβ1-4)Galβ1-3(NeuAcα2-6)GalNAcol |
| Hex2HexNAc3deHex2 | Galβ1-(Fuc)GlcNAcβ1-3(Fucα1-2Galβ1-4GlcNAcβ1-6)GalNAcol |
| Hex2HexNAc3deHex2 | Fucα1-2Galβ1-4GlcNAcβ1-3(Fucα1-2Galβ1-4GlcNAcβ1-6)GalNAcol |
| Hex1HexNAc1deHex1 | Fucα1-2(GalNAcα1-3)Galol |
| Hex1HexNAc4deHex1Sul1 | GlcNAcβ1-3[GalNAcα1-3(Fucα1-2)Galβ1-(6S)GlcNAcβ1-6]GalNAcol |
| NeuAc1Hex2HexNAc2 | NeuAcα2-3Galβ1-3(Galβ1-4GlcNAcβ1-6)GalNAcol |
| NeuAc1Hex2HexNAc3deHex1Sul1 | NeuAcα2-3Galβ1-3[GalNAcα1-3(Fucα1-2)Gα1-(6S)GlcNAcβ1-6]GalNAcol |
| Hex2HexNAc3deHex1 | Galβ1-3[Fucα1-2(GalNAcα1-3)Galβ1-4GlcNAcβ1-6]GalNAcol |
| NeuGc1Hex3HexNAc2 | Galα1-3Galβ1-3(NeuGcα2-3Gal β1-4GlcNAcβ1-6)GalNAcol |
| Hex2HexNAc3deHex1Sul1 | Galβ1-3GlcNAcβ1-3[Fucα1-2Galβ1-4(6S)GlcNAcβ1-6]GalNAcol |
| NeuAc1Hex2HexNAc2Sul1 | NeuAcα2-3Galβ1-3[Galβ1-4(6S)GlcNAcβ1-6]GalNAcol |
| NeuAc1Hex4HexNAc4deHex2Sul1 | |
| NeuGc1Hex2HexNAc2deHex1 | NeuGcα2-3Galβ1-3(Fucα1-2Galβ1-4GlcNAcβ1-6)GalNAcol |
| NeuAc1NeuGc1Hex1HexNAc1 | NeuAcα2-3Galβ1-3(NeuGcα2-6)GalNAcol |
| NeuGc1Hex2HexNAc3deHex1Sul1 | GlcNAcα1-4Galβ1-3[NeuGcα2-6Gα1-(Fuc)(6S)GlcNAcβ1-6]GalNAcol |
| NeuGc1Hex2HexNAc2Sul1 | NeuGcα2-3Galβ1-3[Galβ1-4(6S)GlcNAcβ1-6]GalNAcol |
| NeuAc1Hex2HexNAc2deHex1 | NeuAcα2-3Galβ1-3(Fucα1-2Galβ1-4GlcNAcβ1-6)GalNAcol |
| Hex2HexNAc3 | GlcNAcα1-4Galβ1-3(Galβ1-4GlcNAcβ1-6)GalNAcol |
| NeuGc2Hex1HexNAc2 | NeuGcα2-3(GalNAcβ1-4)Galβ1-3(NeuGcα2-6)GalNAcol |
| Hex1HexNAc3deHex2Sul1 | 6SGlcNAcβ1-3[Fucα1-2Galβ1-(Fucα1-)GlcNAcβ1-6]GalNAcol |
| NeuGc1Hex2HexNAc3 | NeuGcα2-3Galβ1-3(GlcNAcα1-4Galβ1-4GlcNAcβ1-6)GalNAcol |
| HexNAc3Sul1 | 6SGlcNAcβ1-3(GlcNAcβ1-6)GalNAcol |
| NeuAc1Hex2HexNAc2deHex1Sul1 | NeuAcα2-3Galβ1-3[Fucα1-2Galβ1-4(6S)GlcNAcβ1-6]GalNAcol |
| Hex1HexNAc4deHex1 | GlcNAcβ1-3[GalNAcα1-3(Fucα1-2)Galβ1-3GlcNAcβ1-6]GalNAcol |
| Hex1HexNAc1deHex1 | Fucα1-2Galβ1-4GlcNAcol |

The glycopeptide formulation was subjected to beta-elimination and the released glycan profile analyzed via LC-MS/MS and all the 101 glycan structures displayed in the table by there: molecular weight, composition, annotated structure, abundance as percentage of the area under the curve.

In-depth comparison of the glycans in GNU101 to those in the MC38, 4T1 and YUMM1.7 tumor models identified high similarities in truncated core-1 and core-2 glycans as well as 9 unique glycan epitopes present in YUMM1.7 melanoma cells and 11 glycan epitopes in MC38 cells (FIG. 1C-1D). Notably, many of these glycan motifs have been identified to engage lectin receptors for instance on dendritic cells (FIG. 1D). See Lists 2-4 below.

List 2: Melanoma-model YUMM1.7 tumor glycocode

| Composition | Putative structures |
|---|---|
| NeuAc2Hex2HexNAc2 | NeuAcα2-3Galβ1-3(NeuAcα2-3Galβ1-4GlcNAcβ1-6)GalNAcol |
| NeuGc2Hex2HexNAc2 | NeuGcα2-3Galβ1-3(NeuGcα2-3Galβ1-4GlcNAcβ1-6)GalNAcol |
| NeuAc1Hex1HexNAc1 | NeuAcα2-3Galβ1-3GalNAcol |
| NeuGc1Hex1HexNAc1 | NeuGcα2-3Galβ1-3GalNAcol |
| NeuAc2Hex1HexNAc1 | NeuAcα2-3Galβ1-3(NeuAcα2-6)GalNAcol |
| NeuAc1Hex2HexNAc2 | Galβ1-3(NeuAcα2-3Galβ1-4GlcNAcβ1-6)GalNAcol |
| NeuGc2Hex1HexNAc1 | NeuGcα2-3Galβ1-3(NeuGcα2-6)GalNAcol |
| NeuAc1Hex3HexNAc2 | NeuAcα2-3Galβ1-3(Galα1-3Galβ1-4GlcNAcβ1-6)GalNAcol |
| NeuAc1NeuGc1Hex3HexNAc3 | |
| NeuAc2Hex3HexNAc3 | NeuAcα2-3Galβ1-3(NeuAcα2-6Galβ1-4GlcNAcβ1-3Galβ1-4GlcNAcβ1-6)GalNAcol |
| NeuGc1Hex2HexNAc2 | Galβ1-3(NeuGcα2-3Galβ1-4GlcNAcβ1-6)GalNAcol |
| Hex3HexNAc2 | Galβ1-3(Galα1-3Galβ1-4GlcNAcβ1-6)GalNAcol |
| NeuGc1Hex3HexNAc2 | Galα1-3Galβ1-3(NeuGcα2-3Galβ1-4GlcNAcβ1-6)GalNAcol |
| NeuAc2Hex3HexNAc3 | NeuAcα2-3Galβ1-3(NeuAcα2-3Galβ1-4GlcNAcβ1-3Galβ1-4GlcNAcβ1-6)GalNAcol |
| Hex1HexNAc2deHex1Sul1 | Fucα1-2Galβ1-3(6SGlcNAcβ1-6)GalNAcol |
| NeuAc1Hex2HexNAc2 | NeuAcα2-3Galβ1-4GlcNAcβ1-3Galβ1-3GalNAcol |
| NeuAc2Hex2HexNAc2Sul1 | NeuAcα2-3Galβ1-3[NeuAcα2-3Galβ1-3(6S)GlcNAcβ1-6]GalNAcol |
| NeuGc2Hex3HexNAc3 | NeuGcα2-3Galβ1-4GlcNAcβ1-3Galβ1-3(NeuGcα2-3Galβ1-4GlcNAcβ1-6)GalNAcol |
| Hex2HexNAc2 | Galβ1-3(Galβ1-4GlcNAcβ1-6)GalNAcol |
| NeuGc1Hex2HexNAc2 | NeuGcα2-3Galβ1-4GlcNAcβ1-3Galβ1-3GalNAcol |
| NeuGc2Hex5HexNAc4 | NeuGcα2-6Galβ1-4GlcNAcβ1-2Manα1-3(NeuGcα2-6Galβ1-4GlcNAcβ1-2Manα1-6)Manβ1-4GlcNAcβ1-4GlcNAcol |
| Hex1HexNAc1deHex1 | Fucα1-2Galβ1-3GalNAcol |
| NeuAc1Hex2HexNAc2 | NeuAcα2-3Galβ1-3(Galβ1-4GlcNAcβ1-6)GalNAcol |
| NeuAc2Hex2HexNAc2Sul1 | NeuAcα2-Galβ1-3[NeuAcα2-Galβ1-3(6S)GlcNAcβ1-6]GalNAcol |
| Hex1HexNAc2Sul1 | |
| Hex9HexNAc2 | Manα1-2Manα1-2Manα1-3[Manα1-2Manα1-3(Manα1-2Manα1-6)Manα1-6]Manβ1-4GlcNAcβ1-4GlcNAcol |
| NeuAc1Hex1HexNAc2Sul1 | NeuAcα2-3Galβ1-3[(6S)GlcNAcβ1-6]GalNAcol |
| NeuGc2Hex2HexNAc2Sul1 | NeuGcα2-3Galβ1-3[NeuGcα2-3Galβ1-4(6S)GlcNAcβ1-6]GalNAcol |
| Hex8HexNAc2 | Manα1-2Manα1-2Manα1-3[Manα1-3(Manα1-2Manα1-6)Manα1-6]Manβ1-4GlcNAcβ1-4GlcNAcol |
| HexNAc2Sul1 | 6SGlcNAcβ1-6GalNAcol |
| Hex6HexNAc2 | Manα1-2Manα1-3[Manα1-3(Manα1-6)Manα1-6]Manβ1-4GlcNAcβ1-4GlcNAcol |
| NeuGc1Hex1HexNAc2Sul1 | NeuGcα2-3Galβ1-3(6SGlcNAcβ1-6)GalNAcol |
| NeuAc1Hex1HexNAc1 | Galβ1-3(NeuAcα2-6)GalNAcol |
| NeuGc1Hex2HexNAc2 | NeuGcα2-3Galβ1-3(Galβ1-4GlcNAcβ1-6)GalNAcol |

List 3: CRC-model MC.38 tumor glycocode

| Composition | Putative structures |
|---|---|
| NeuAc2Hex2HexNAc2 | NeuAcα2-3Galβ1-3(NeuAcα2-3Galβ1-4GlcNAcβ1-6)GalNAcol |
| NeuAc1NeuGc1Hex2HexNAc2 | NeuGcα2-3Galβ1-3(NeuAcα2-3Galβ1-4GlcNAcβ1-6)GalNAcol |
| NeuAc1Hex1HexNAc1 | NeuAcα2-3Galβ1-3GalNAcol |
| NeuAc2Hex1HexNAc1 | NeuAcα2-3Galβ1-3(NeuAcα2-6)GalNAcol |
| NeuAc1Hex2HexNAc2 | Galβ1-3(NeuAcα2-3Galβ1-4GlcNAcβ1-6)GalNAcol |
| NeuAc1Hex3HexNAc2 | NeuAcα2-3Galβ1-3(Galα1-3Galβ1-4GlcNAcβ1-6)GalNAcol |
| NeuGc2Hex2HexNAc2 | NeuGcα2-3Galβ1-3(NeuGcα2-3Galβ1-4GlcNAcβ1-6)GalNAcol |
| NeuAc1Hex2HexNAc2 | NeuAcα2-3Galβ1-3(Galβ1-4GlcNAcβ1-6)GalNAcol |
| NeuGc1Hex1HexNAc1 | NeuGcα2-3Galβ1-3GalNAcol |
| NeuAc1NeuGc1Hex1HexNAc1 | NeuGcα2-3Galβ1-3(NeuAcα2-6)GalNAcol |
| Hex3HexNAc2 | Galβ1-3(Galα1-3Galβ1-4GlcNAcβ1-6)GalNAcol |
| Hex2HexNAc2 | Galβ1-3(Galβ1-4GlcNAcβ1-6)GalNAcol |
| NeuAc1Hex1HexNAc1 | Galβ1-3(NeuAcα2-6)GalNAcol |
| NeuGc1Hex1HexNAc1 | Galβ1-3(NeuGcα2-6)GalNAcol |

List 4: βreast cancer-model 4T1 tumor glycocode

| Composition | Putative structures |
|---|---|
| NeuAc2Hex1HexNAc1 | NeuAcα2-3Galβ1-3(NeuAcα2-6)GalNAcol |
| NeuAc2Hex2HexNAc2 | NeuAcα2-3Galβ1-3(NeuAcα2-3Galβ1-4GlcNAcβ1-6)GalNAcol |
| NeuAc1NeuGc1Hex1HexNAc1 | NeuGcα2-3Galβ1-3(NeuAcα2-6)GalNAcol |
| NeuAc1Hex1HexNAc1 | NeuAcα2-3Galβ1-3GalNAcol |
| NeuGc2Hex1HexNAc1 | NeuGcα2-3Galβ1-3(NeuGcα2-6)GalNAcol |
| NeuGc1Hex1HexNAc1 | NeuGcα2-3Galβ1-3GalNAcol |

The YUMM1.7, MC38 and 4T1 tumors were harvested at the end of the respective experiments and subjected to beta-elimination and the released glycan profile analyzed via LC-MS/MS and all the 101 glycan structures displayed in the table by there: molecular weight, composition, annotated structure, abundance as percentage of the area under the curve. The respective ‘abundance’ is depicted in FIG. 1D.

### Administration of GNU101 Reduces Tumor Growth.

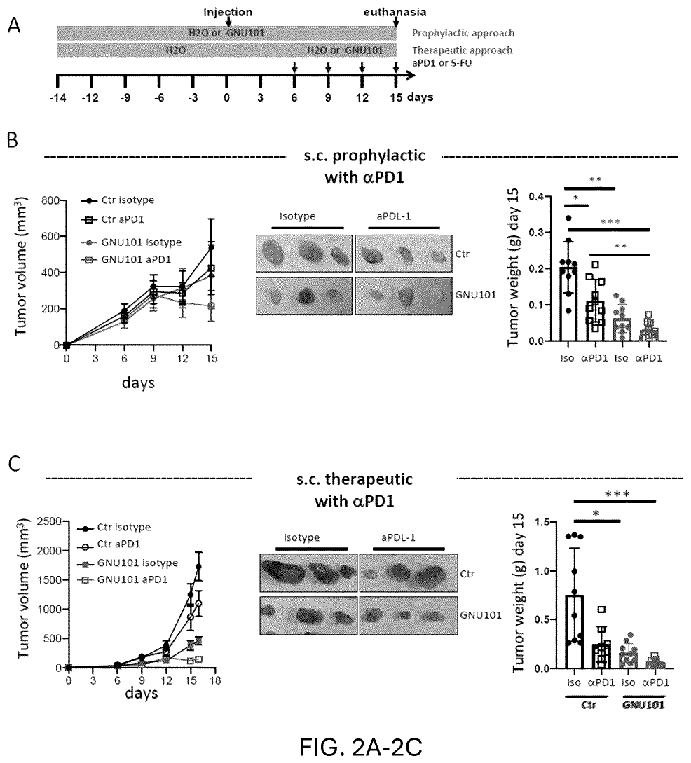

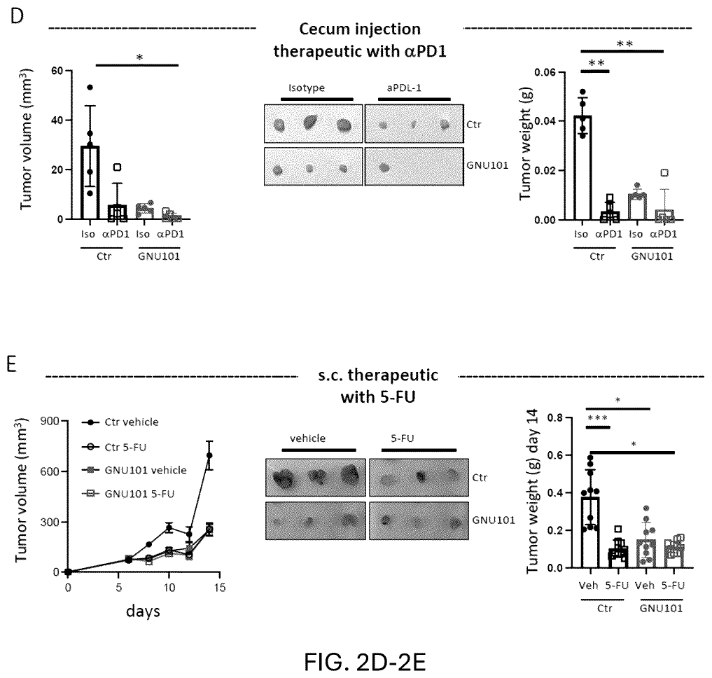

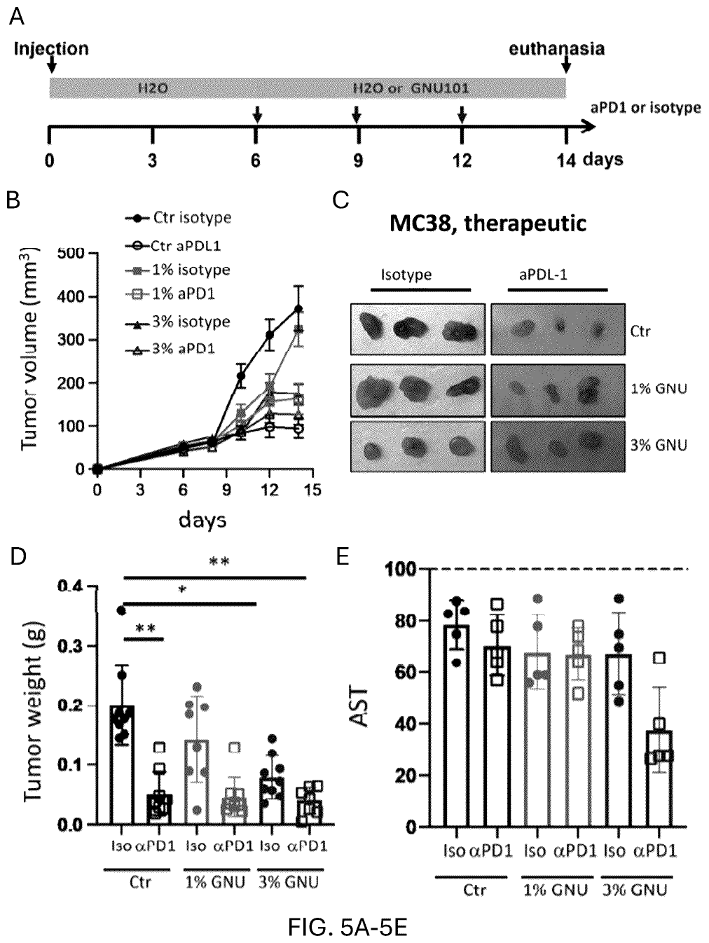

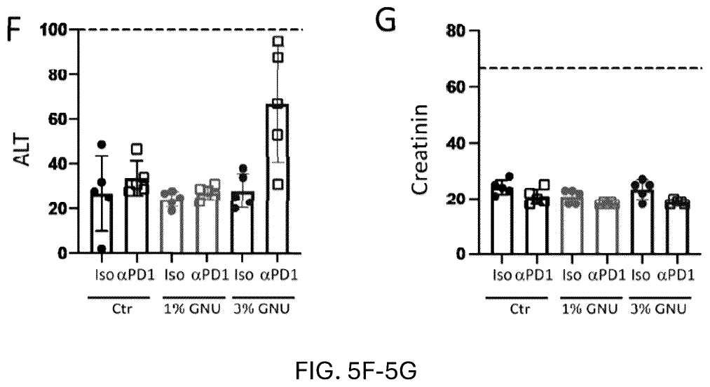

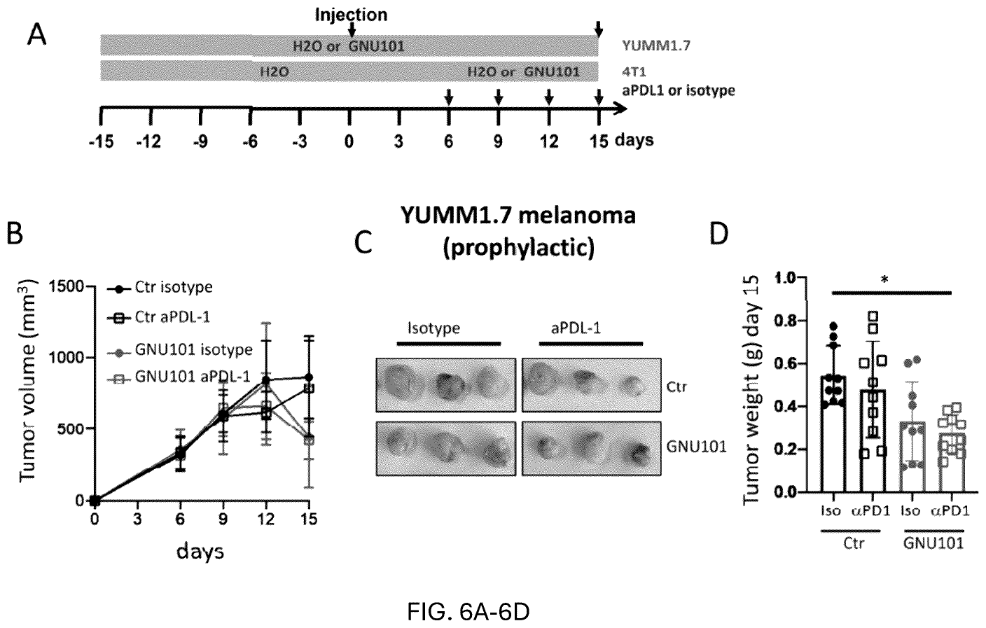

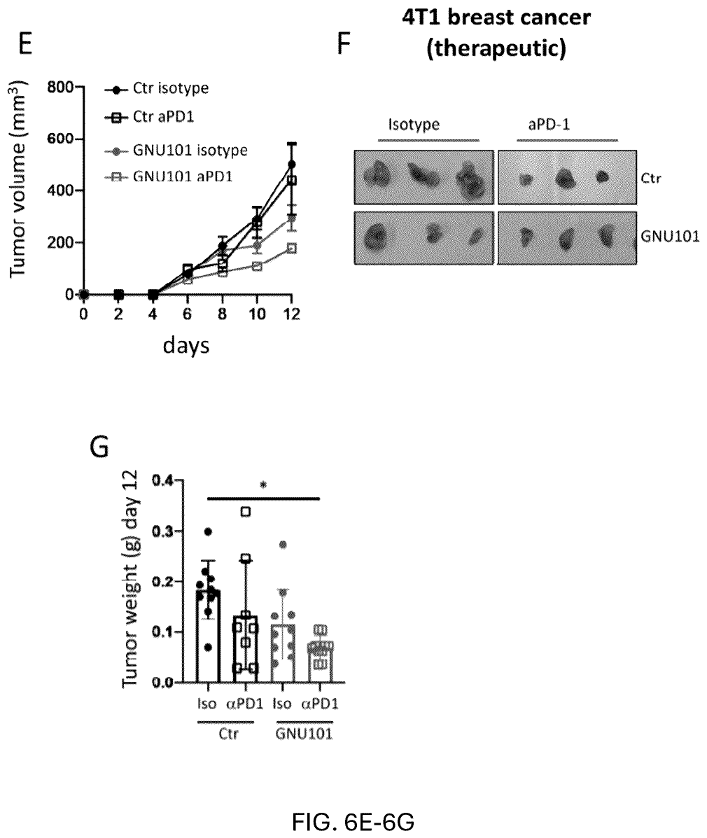

In a subcutaneous tumor model (FIG. 2A) using MC38 CRC cells, administration of GNU101 to the drinking water at an optimal dosing of 3% w/v resulted in significantly reduced tumor growth (FIGS. 2B&2C, FIGS. 5A-5G), both, in a prophylactic (starting treatment 2 weeks prior to tumor cell injection) and a therapeutic (starting treatment 6 days after tumor cells injection) setting (FIG. 2A-C, FIGS. 5A-5G). In both approaches, a single treatment with GNU101 or the checkpoint inhibitor anti-PD1 showed efficacy, while the combination of GU101 with anti-PD1 further enhanced the anti-tumor effect (FIGS. 2B & 2C). Similar effects were observed in the orthotopic MC38 cecum injection model (FIG. 2D). Combination therapy was also effective in subcutaneously injected YUMM1.7 and 4T1 cell-derived tumors, which respond poorly to anti-PD1 treatment (FIGS. 6A-6F). While the combination of GNU101 with anti-PD1 had an additive anti-tumor effect, the combination with the chemotherapeutic 5-FU did not, however, GNU101 alone was as efficient in reducing tumor growth as 5-FU (FIG. 2E). Taken together, these findings indicate that administration of GNU101 reduces tumor growth, with a significant additive effect when GNU101 was combined with anti-PD1.

### GNU101 Promotes T Cell Activation.

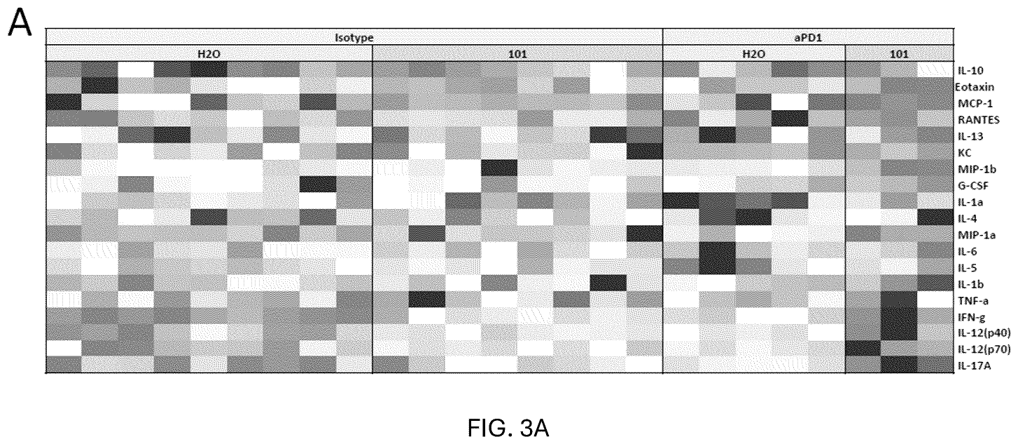

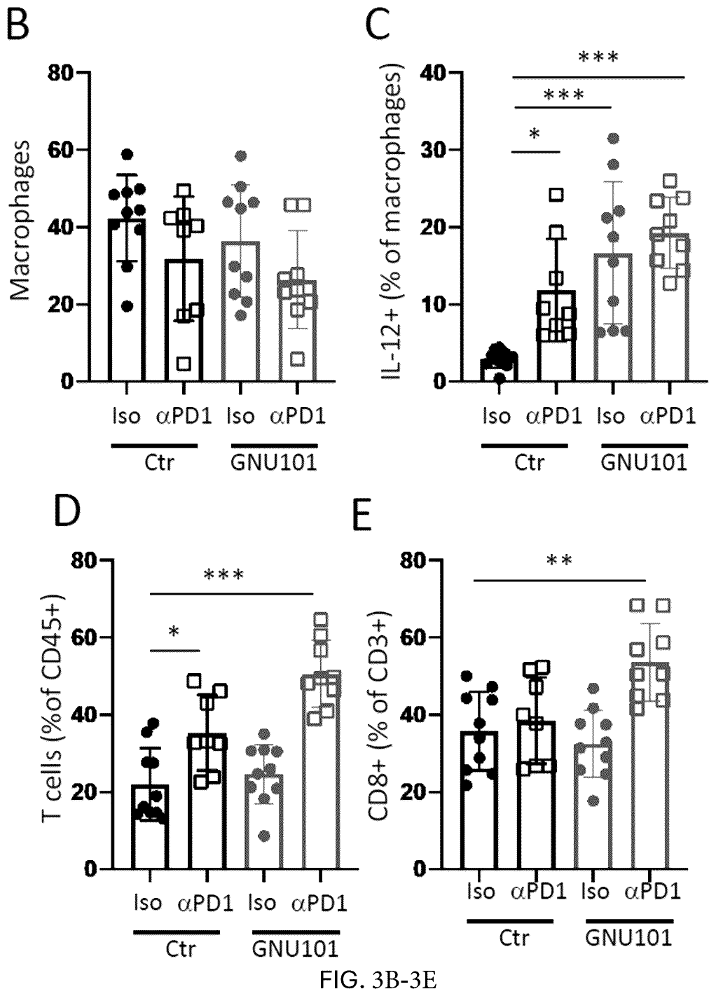

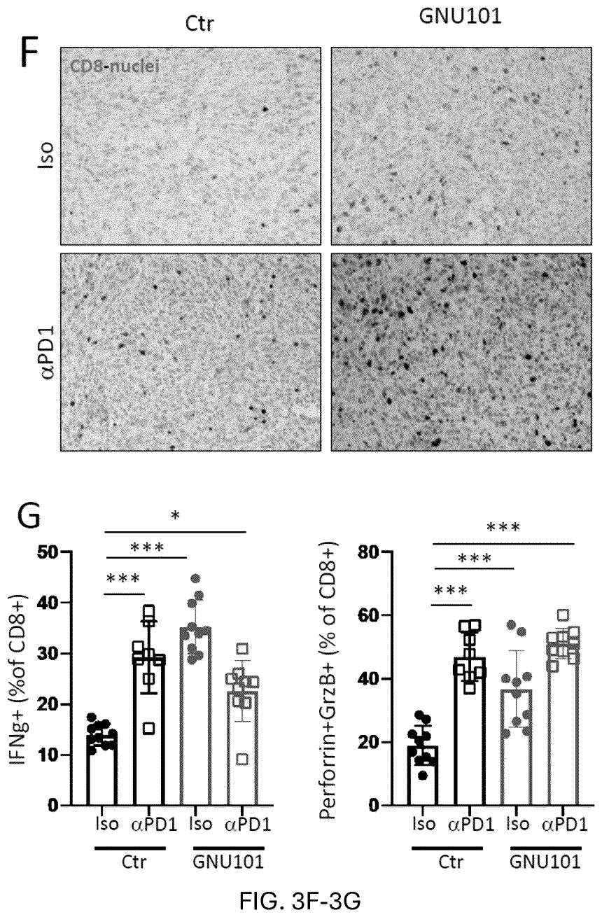

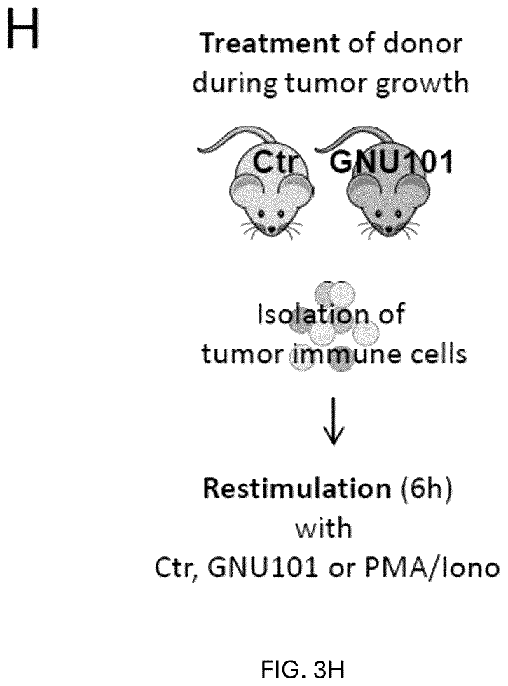

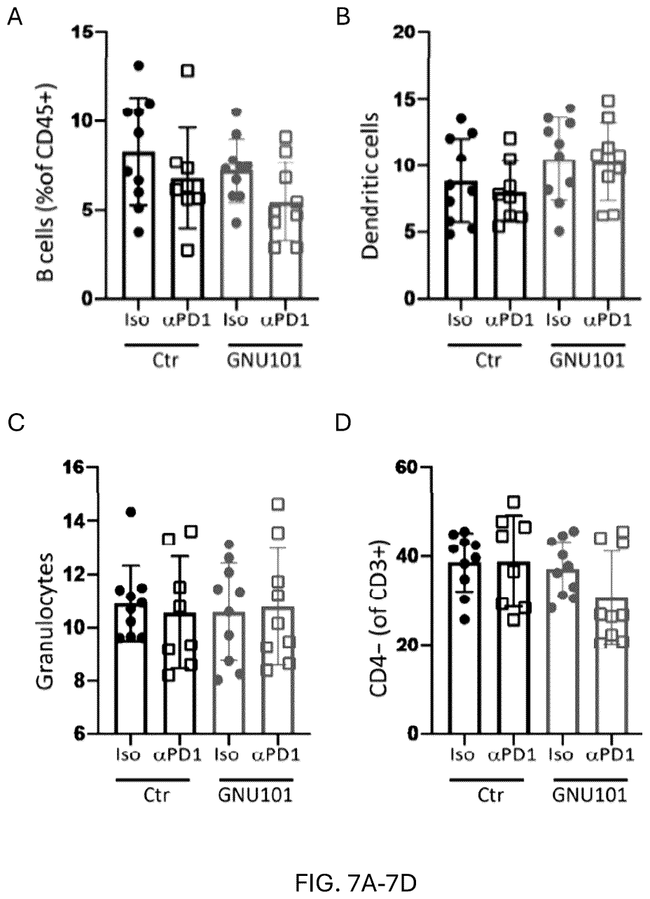

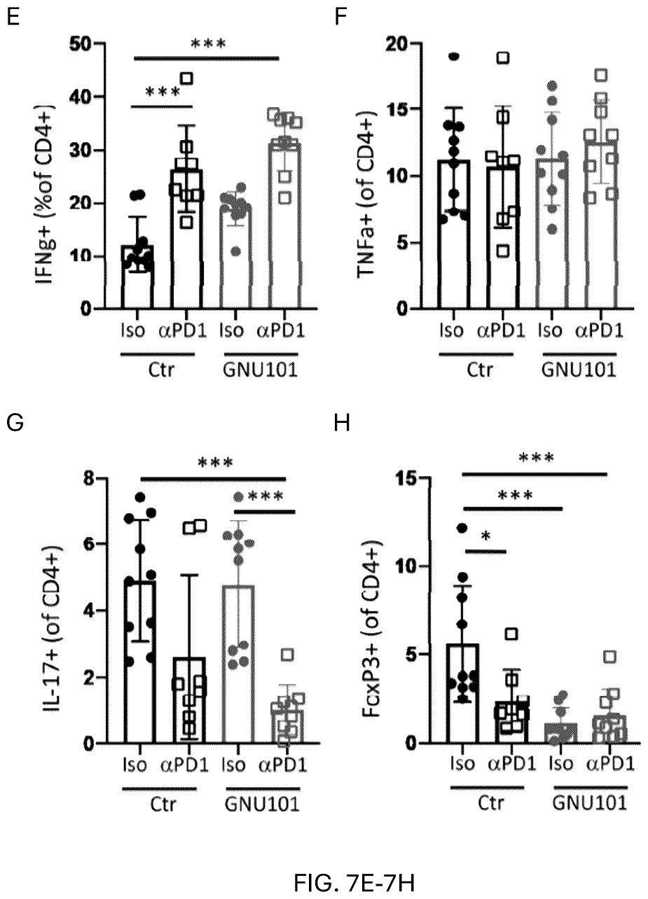

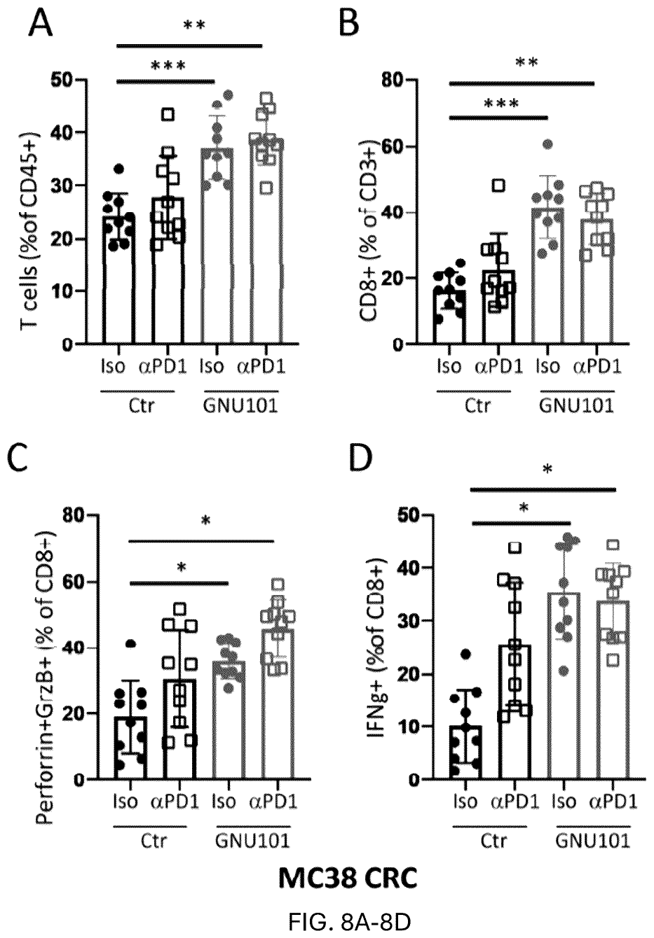

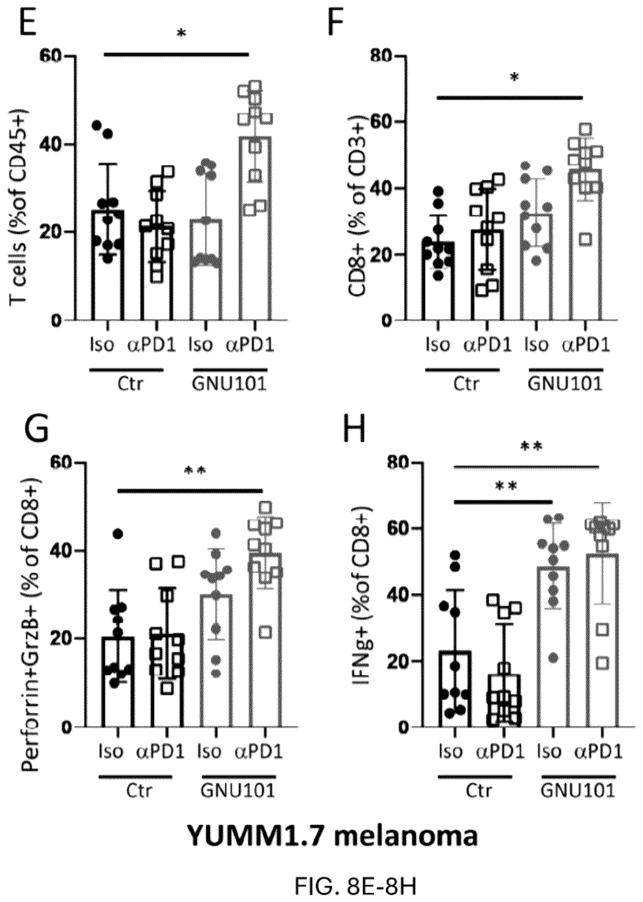

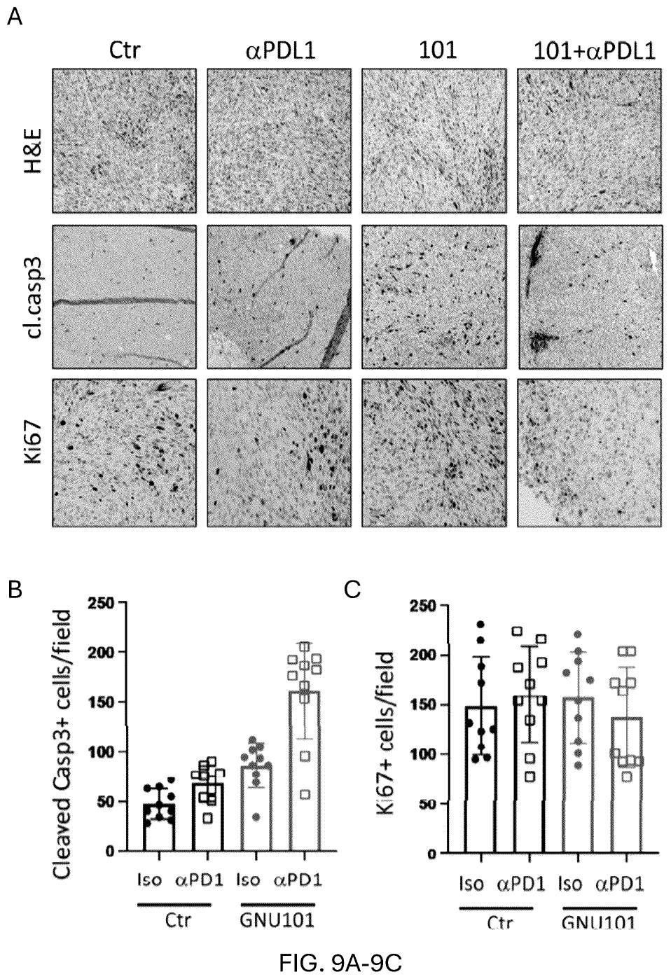

Cytokines associated with a pro-inflammatory immune response (i.e. IL-1β, IL-12, TNFα, IFNγ, IL-17) were significantly increased in tumors from GNU101-treated mice, while regulatory molecules, such as IL-10, and molecules involved in macrophage recruitment (e.g. MCP1, MIP1α) were reduced (FIG. 3A). The proportion of macrophages among all immune cells was not affected, however, the fraction of macrophages that produced IL-12 was increased upon administration of GNU101 or GNU101 in combination with anti-PD1 (FIGS. 3B&3C). The relative abundance of most immune cell subsets was not affected by GNU101 treatment or a combination of GNU101 and anti-PD1 (FIGS. 7A-7D), but T cells, and especially CD8+ T cells were increased following treatment with GNU101 in combination with anti-PD1 (FIGS. 3D-3F, FIGS. 7E-7H). GNU101 administration promoted IFNγ and GranzymeB/Perforin production from CD8+ T cells with compounding effects in the combination treatment with anti-PD1 (FIG. 3G). Similar effects on the immune cell landscape were observed in the prophylactic models using MC38 and YUMM1.7 cells (FIGS. 8A-8H). Histology revealed that more cells were positive for the apoptosis marker cleaved caspase 3, while the overall number of Ki67+ (proliferating) cells was unchanged (FIGS. 9A-9C). Thus, administration of GNU101 promotes an overall pro-inflammatory immune response, the generation or accumulation of cytotoxic T cells in the tumor microenvironment, and ultimately reduced tumor size. When T cells were isolated from tumors previously treated with GNU101 and subsequently restimulated with GNU101 in vitro (FIG. 3H), significantly more T cells were activated as compared to T cells isolated from GNU101-naive mice (FIGS. 3H&3J). Consistent with this result, GNU101 only provoked a significant IFNγ response in T cells from tumors of mice that had been treated with GNU101 (FIG. 3J). This suggests that the administration of GNU101 promotes the generation or expansion of glycopeptide-specific T cells. Transfer of T cells from tumors of GNU101-treated mice prevents tumor growth.

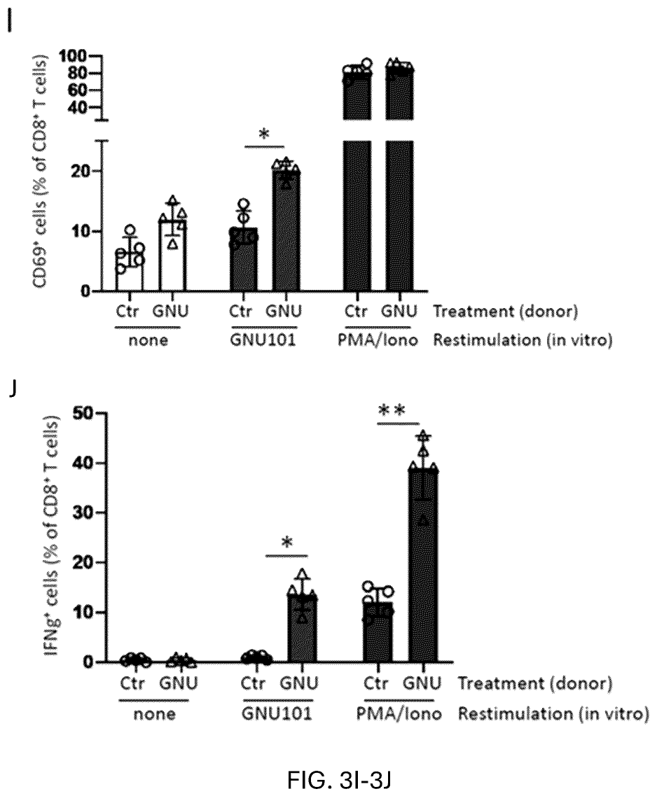

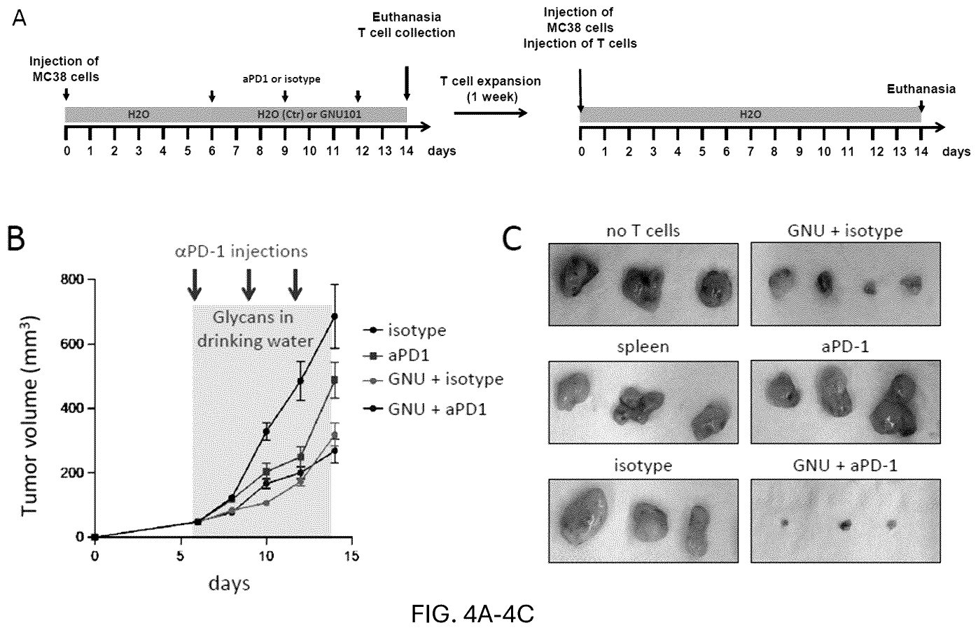

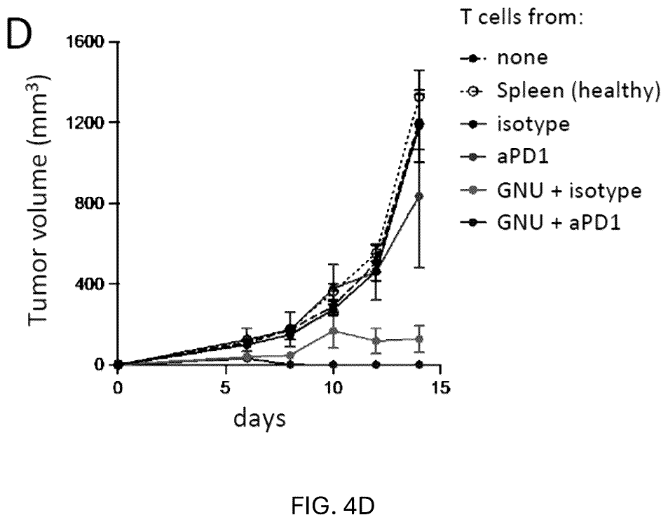

When T cells were isolated from tumors of GNU101 or GNU101 combined with anti-PD-1 therapeutically treated mice and adoptively transferred into treatment naïve hosts at the timepoint of tumor establishment (FIG. 4A), they were able to significantly reduce tumor growth (FIG. 4B), resulting in significantly smaller tumors at the end of the experiment. In the naïve mice that received T cells from GNU101 with anti-PD-1 treated mice, tumor growth was almost completely abolished (FIGS. 4C-4D). In contrast, T cells isolated from the spleen of healthy mice, or from tumors of mice that did not receive GNU101 did not affect tumor growth (FIGS. 4A-4D).

### Discussion

The data presented in this disclosure demonstrated that oral administration of the novel glycopeptide formulation GNU101, which mimics the tumor glyco-code, provokes a strong, highly specific anti-tumor T cell response. While T cell activation was robust and mediated a long-lasting anti-tumor effect that was transmissible into animals that were never exposed to GNU101. Upon T cell transfer, the response was highly tumor-specific and did not result in toxic effects or aberrant immune responses in other organs. By provoking such a distinct, tumor-specific response this formulation has clear benefits over approaches that result in the non-specific killing of fast proliferating cells (e.g. cytostatic drugs) or general immune activation (i.e. checkpoint inhibitor treatment) that frequently provoke severe side effects (Suijkerbuijk et al., 2024).

Of note, given the oral route of administration of the compound, it is unlikely that GNU101 activates T cells directly (Rodríguez et al., 2018). Thus, an intermediate cell or mechanisms triggered by the presence of GNU101 in the intestine must mediate anti-tumor T cell activation. It has been demonstrated that glycans trigger lectin receptors on antigen-presenting cells (APC), promoting their maturation and ability to present antigens (Reily et al., 2019; Sosa Cuevas et al., 2023). DCs (Van Gisbergen et al., 2005) recognize, process and present glycan antigens to T cells and promote glycan-specific T cell responses (Apostolopoulos et al., 2003; Parker et al., 2021; Sun et al., 2020). Notably, in clinical trials where fecal microbiota transfer (FMT) from checkpoint inhibitor therapy responders to non-responders was used to induce response to ICI therapy, the approach was most successful in those patients where innate immune cell accumulation and reduction in immune-suppressive myeloid cells in the intestine was observed (Davar et al., 2021) Hence, innate intestinal immune cells seem to be key to enabling robust anti-tumor immunity. Thus, it is likely that GNU101, via activation of APCs in the intestine, and subsequent presentation of glycopeptides on DCs, drives the activation and/or expansion of glycopeptide-specific T cells. Since the GNU101 glycopeptides mimic those on tumors, T cells activated by GNU101-exposed APCs subsequently react to these structures on the tumors and promote anti-tumor immunity.

In addition to its strong effect on T-cell activity as a stand-alone treatment, there was a clear additive effect in T-cell recruitment when GNU101 was combined with anti-PD-1 checkpoint inhibition. This indicates a complementary mechanism of GNU101 and PD1 inhibition, further strengthening our hypothesis that GNU101 does not promote general, unspecific T cell activation but could act via glycopeptide display by APCs that subsequently provoke a specific T-cell immune response against glycopeptides on the tumors which are mimicked in our formulation. In turn, anti-PD1 relieves unspecific suppressive signals that promote T cell exhaustion and anergy (Johnson et al., 2022) Thus, when GNU101 glycopeptides are combined with anti-PD1 treatment, the GNU101-induced tumor-specific T cells are less prone to exhaustion which ultimately further increases their effectiveness in combating the tumor.

Oral immunization with polysaccharide antigens typically results in T cell-independent, B-cell-driven immunity, which is beneficial for immunization against mucosal pathogens but might be suboptimal to drive T-cell-mediated immunity against tumors (Lavelle & Ward, 2022a), although it had been suggested that oral vaccination might be effective against tumors (Sandoval et al., 2013). Indeed, immunization with single glycan motifs was shown to provoke strong immune responses but was suboptimal to mediate robust anti-tumor immunity (Lavelle & Ward, 2022a, 2022b). In contrast, our glycopeptide formulation, consisting of a multitude of glycans present on tumor cells, was able to provoke robust anti-tumor T cell responses. It is likely that mimicking the pleiotropy of epitopes found on tumor cells is necessary to overcome the tolerogenic nature of mucosal-delivered antigens and that the delivery of a large part of the “tumor glyco-code” indeed represents the strength of our product and is the key to induce a robust, highly specific and long-lasting T cell anti-tumor immunity. Thus, we here describe a highly efficient novel therapeutic vaccine to boost anti-tumor immunity for those CRC patients who respond poorly to current treatment regimens.

### Methods

Glycan content A mixture of 10 mg of GNU101 powder and 2 ml of 2M trifluoroacetic acid (Sigma-Aldrich, #T6508-100 ml) was incubated at 100° C. for 4 h to allow the GNU101 to be hydrolyzed and release glycan monosaccharide-building blocks. Further dilution to 500 mg/L and filtration through a 0.22 μm filter (Durapore, #GVWP04700). Subsequently, 10 μl was injected per auto-sampler to a Thermo Scientific Dionex ICS-6000 PAD (Ion-exchange Chromatography System with Pulsed Amperometric Detection) system. Monosaccharides (L-fucose, N-acetyl galactosamine, N-acetyl glucosamine, galactose, glucose and mannose) were separated on a Thermo Scientific Dionex CarboPac PA20 column (150 mm×3 mm) using a multi-step gradient KOH aqueous eluent (10 mM to 100 mM by EGC 500 cartridge). The concentrations were determined according to the 6 monosaccharides standards: L-fucose (USP, #1286606-200 mg), Galactosamine hydrochloride (Supelco, #PHR2042-200 mg), Glucosamine hydrochloride (Supelco, #PHR1199-500 mg), galactose (Supelco, #PHR1206-500 mg), glucose (Supelco, #PHR1000-1g) and mannose (Supelco, #PHR2044-1g). Data acquisition and processing were conducted with Chromeleon software (Version 7).

Glycan Profile The glycan profiles were analyzed as described (Karlsson et al., 2009), the tumor samples were homogenized in 0.5 ml 7 M urea (Sigma-Aldrich, #U5128), 2 M thiourea (Sigma-Aldrich, #T8656), 40 mM Tris (tris(hydroxymethyl) aminomethane) (Millipore, #9210-OP) and 1% CHAPS (Millipore, #220201) containing 5 mM PMSF (phenylmethylsulfonyl fluoride) (Sigma-Aldrich, #78830) and 25 mM DTT (dithiothreitol) (Sigma-Aldrich, #D0632). The samples were extracted in the cold room overnight with rotation. After centrifugation at full speed, add 27 ml 500 mM IAA (indole-3-acetic acid) (Millipore, #1.00353) to the supernatant and keep in the dark at room temperature of 45 min. After alkylation, the sample was dot-blotted to PVDF (polyvinylidene fluoride) membrane (ca. 15 ml/well). After staining with alcian blue (Sigma-Aldrich, #TMS-010) and washing with methanol, the whole membrane was blocked with 10 ml 1% PVP (polyvinyl pyrrolidone 40,000, w/w) in 50% methanol for 10 min with shake, followed by 3×5 min washing with water. The dots were excised and transferred to a tube for glycan release. O-Glycans from tumor samples and from GNU101 were then released by β-elimination in 50 mM NaOH (Sigma-Aldrich, #S5881) and 0.5M NaBH₄ (Sigma-Aldrich, #213462). The samples were incubated at 50° C. for 16 h. Then the samples were slowly neutralized with concentrated acetic acid (HOAc). Aliquots (20 μl) of the samples were desalted using cation exchange resin (AG50W×8) packed onto a ZipTip C18 tip. After drying the samples in SpeedVac, 50 μl 1% HOAc in methanol was added repeatedly to remove residual borate by evaporation. The resultant glycans were then analyzed by using LC-MS/MS.

Released glycans were resuspended in water and analyzed by liquid chromatograph-electrospray ionization tandem mass spectrometry (LC-ESI/MS). The glycans were separated on a column (10 cm×250 μm) packed in-house with 3 μm porous graphite particles (Hypercarb, Thermo-Hypersil, Runcorn, UK). The oligosaccharides were injected on to the column and eluted with an acetonitrile gradient (Buffer A, 10 mM ammonium bicarbonate; Buffer B, 10 mM ammonium bicarbonate in 80% acetonitrile); Buffer C: 0.1% HOAc. The gradient (0-45% Buffer B) was eluted for 30 min, followed by 8 min with 100% Buffer B, followed by 10 min with 0.1% HOAc, and equilibrated with Buffer A in the next 15 min. A 40 cm×50 μm i.d. fused silica capillary was used as transfer line to the ion source.

The samples were analyzed in negative ion mode on a LTQ linear ion trap mass spectrometer (Thermo Electron, San José, CA), with an IonMax standard ESI source equipped with a stainless steel needle kept at-3.5 kV. Compressed air was used as nebulizer gas. The heated capillary was kept at 270° C., and the capillary voltage was-50 kV. Full scan (m/z 380-2000, two microscan, maximum 100 ms, target value of 30,000) was performed, followed by data-dependent MS2 scans (two microscans, maximum 100 ms, target value of 10,000) with normalized collision energy of 35%, isolation window of 2.5 units, activation q=0.25 and activation time 30 ms). The threshold for MS2 was set to 300 counts. Data acquisition and processing were conducted with Xcalibur software (Version 2.0.7).

Animals. Animal experiments were performed according to Swiss animal welfare legislation and approved by the local veterinary office (Veterinary Office of the Canton Zurich, license number ZH018/2021). Male and female C57BL/6JRj and Balb/C mice were purchased from Janvier Labs (France) and kept in specific-pathogen-free (SPF) conditions with 12: 12h light: dark cycle and food and water ad libitum.

Tumor cell injection. For subcutaneous injection experiments, MC38, 4T1, or YUMM1.7 were suspended in culture medium (DMEM, high glucose, Gibco #7001567, supplemented with 10% FCS, sodium pyruvate and non-essential amino acids) and 300,000 cells were injected into each flank. Tumor volume was measured every 2 days using the ellipsoid formula 4/3*3.14*Length/2*(Width/2) 2. Mice were terminated approx. two weeks after tumor cell injection or when the volume exceeded 1.5 cm³ or if wounds developed on the tumor. For cecum injections, 300,000 cells were suspended in culture medium, mixed 1:1 in Matrigel (Corning, #FAL354263), and injected into the cecum wall during laparotomy. Mice were euthanized 2 weeks after tumor cell injection.

Anti-PD1 and 5-FU treatment. For experiments with anti-PD1, mice were injected with 100 μg anti-PD1 antibody (BioXcell, #BP0273, clone 29F.1A12) or an isotype control antibody (BioXcell, #BP0089, clone 2A3) every three days starting on day 6 after tumor cell injection. For experiments, mice were injected with 1 mg 5-FU (Sigma Aldrich/Merck #F6627-1G) or vehicle (20% DMSO in water) every three days starting on day 6 after tumor cell injection.

GNU101 supplementation. GNU101 was dissolved in drinking water at a concentration of 3% or 1% (w/v) and sterile filtered through 0.22 μm pore filters

(TPP, #7.995000). Drinking water was replaced every 36 h to prevent bacterial growth. For prophylactic treatment, the mice received GNU101 supplemented water ad libitum starting 2 weeks prior to tumor cell injection, for therapeutic treatment, the mice received GNU101 supplemented water starting on day 6 after tumor cell injection.

Multiplex cytokine analysis. For cytokine analyses, 0.1 g tumor tissue was homogenized in 100 μl PBS and analyzed using a 23-plex mouse cytokine assay from BioRad (#M60009RDPD) according to the manufacturer's instructions.

Immune cell isolation from tumors. Immune cells were isolated from tumor tissue as described. In brief, tumors were excised, minced into small pieces and incubated in a shaker at 37° C. for 10 min in digestion solution (Hank's Buffered Salt Solution [Sigma-Aldrich, #H4891-10X1L], supplemented with 10% FCS, 0.5 mg/ml Collagenase IV [Sigma Aldrich, #9263-1G] and 0.05 mg/ml DNase I [Sigma Aldrich, #10104159001]. The sample was passed through 26G syringes to homogenize, and the cells filtered through 40 μm cell strainers.

Restimulation of immune cells. For restimulation, 1×10⁶ immune cells from the tumor were seeded in 24 well plates in 1 ml culture media (RPMI [life technologies, #11835-063] and 5% FCS) supplemented with 0.1% GNU101 and 1 μg/ml Brefeldin A (Thermo Fisher Scientific, #ACR29714-0250) or with 1 μg/ml Ionomycin (Sigma Aldrich, #13909-1ML), 20 ng/ml PMA (Enzo, #BML-PE162-0001) and 1 μg/ml Brefeldin A and incubated for 6 h prior to analysis by flow cytometry.

Flow cytometry. For flow cytometry, cells were isolated from the tumors as described above. For measurement of cytokine production, half of the cells were restimulated using 1 μg/ml Ionomycin, 20 ng/ml PMA and 1 μg/ml Brefeldin A for 3.5 h, while the other half was used for direct staining. For direct staining, cells were incubated for 20 min. with antibody cocktail, washed twice in FACS buffer (PBS containing 2% FCS and 2 mM EDTA) fixed using 1% paraformaldehyde and analyzed using a Cytek Aurora flow cytometer or a Symphony flow cytometer from BD.

Restimulated cells were washed with PBS, incubated with Zombie-NIR viability dye (Biolegend, #423105) and TruStain FcX PLUS (Biolegend, #156603) for 15 min, washed twice in FACS buffer and incubated with antibodies against surface epitopes for 20 min. Cells were washed twice with FACS buffer, fixed for 30 min using the Transcription Factor Buffer Set from eBioscience (#00-5523-00), washed with permeabilization buffer from the kit and incubated for 30 min with antibodies against intracellular/intranuclear antigens. Cells were washed twice with permeabilization buffer and resuspended in FACS buffer for acquisition on a Cytek Aurora or a BD Symphony flow cytometer. The following antibodies were used in this study: anti-mouse CD3-BV785 (clone 17A2, Biolegend, #100232), anti-mouse NK1.1-BV711 (clone PK136, Biolegend, #108745), anti-mouse CD4-BV650 (clone RM4-5, Biolegend #100546), anti-mouse CD45-BV510 (clone 30-F11, Biolegend, #103138), anti-mouse CTLA4-PE-Cy7 (clone UC10-4B9, Biolegend, #106314), anti-B220-PE-Cy5 (clone RA3-6B2, BD, #552772), anti-mouse CD8-CF594 (clone 53-6.7, BD, #562283), anti-mouse Tim3 (clone B8.2C12, Biolegend, #134004), anti-mouse PD1-APC (clone 29F.1A12 Biolegend, #135210), anti-mouse CD80-BUV737 (clone 16-10A1, BD, #612773), anti-mouse CD3-BUV661 (clone 145-2C11, BD, #750638), anti-mouse CD4-BUV563 (clone GK1.5, BD, #612923), anti-mouse NK1.1-BUV395 (clone PK136, BD, #564144), anti-mouse TNFα-BV785 (clone MP6-XT22, Biolegend, #506341), anti-mouse CD11c-PE-Cy5.5 (clone N418, eBioscience, #35-0114-82), anti-mouse CD11b-BV605 (clone M1/70, Biolegend, #101257), anti-mouse B220-BV570 (clone RA3-6B2, Biolegend, #103273), anti-mouse IL-17-BV510 (clone TC11-18H10.1, Biolegend, 506933), anti-mouse CD45-BV480 (clone 30F11, BD, #566095), anti-mouse FoxP3-PacificBlue (clone MF-14, Biolegend, 126410), anti-mouse GranzymeB-PerCPCy5.5 (clone QA16A02 Biolegend, #372212), anti-mouse F4/80-Fitc (clone QA17A29, Biolegend, #157310), anti-mouse IFNγ-PE-Cy7 (clone XMG1.2, eBioscience, #25-7311-82), anti-mouse IL-12-PE (clone 15.6, Biolegend, #505204), anti-mouse MHCII-AlexaFluor700 (clone M5/114.15.2, Biolegend, #107622), anti-mouse Perforin-APC (cloneS16009B, Biolegend, #154404)

Immunohistochemistry. For immunohistochemistry, tissue pieces were collected from the tumors and fixed in 4% formalin solution for 24 h, followed by dehydration in ascending alcohol series, incubation in Histoclear (Chemie Brunschwig, #NADHS-200-1 gal) and paraffin embedding. For immunohistochemistry, 3 μm slides were cut and fixed on cover slides. The slides were rehydrated in descending alcohol series and antigens retrieved by boiling for 30 min in citrate buffer pH6 (Dako, #S169984-2). After washing in PBS, endogenous peroxidases were inactivated with 0.9% H₂O₂ solution and unspecific binding blocked by incubation with 3% BSA for 1 h prior to overnight incubation with anti-mouse CD8 (clone D4W2Z, Cell Signaling, #9894) antibody. On the next day, the slides were washed three times in PBS, incubated for one h with an anti-Rabbit secondary antibody (Vector Laboratories; VC-MP-7401-L050 ImmPRESS™ anti-Rabbit IgG HRP), and stained using the DAB ImmPACT Peroxidase Substrate from Vector Laboratories (#VC-SK-4105-L120). The sections were counterstained for 10 seconds with hematoxylin (Artechemis, #US020), rinsed in tap water and dehydrated in ascending alcohol series before mounting with Pertex (Biosystems Switzerland AG, #41-4012-00). Images were recorded using a light microscope from Zeiss (Zeiss Imager Z2). Quantification was performed using Fiji Image analysis software.

T cell expansion and injection. For in vitro T cell expansion, immune cells were isolated from the tumor tissue as described above. Subsequently, T cells were isolated using a mouse T cell isolation kit from Stemcell Technologies (#19851) following the manufacturer's instructions. T cells were seeded in T cell expansion media (RPMI, 10% FCS, 50 μM β-mercaptoethanol, 50 U/ml IL-2 [Peprotech, #212-12-5UG]) and anti-CD3/anti-CD28 Dynabeads (Thermo Fisher Scientific, #11456D) added at a T cell: beads ratio of 1:1. The expanding T cells were diluted every 2-4 days with fresh T cell expansion media. After one week, T cells were harvested, counted and 4×10⁶ cells injected intraperitoneally to each recipient on the same day as the tumor cell injection. Expanded T cells from one mouse were used for one recipient.

Statistical analysis. When three or more groups were compared, two-tailed Kruskal-Wallis test with Dunn's post-hoc test was performed. When comparing two groups, non-parametric two-tailed Mann Whitney test was used. P values below 0.05 were considered significant.

### Equivalents

Unless otherwise defined, all technical and scientific terms used herein have the same meaning as commonly understood by one of ordinary skill in the art to which this invention belongs.

Throughout this disclosure, various technical publications are referenced by an author or authors name and date. The full bibliographic details of the publication can be found in the reference section, immediately preceding the claims. These are incorporated by reference in their entireties.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present disclosure has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The embodiments have been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

### REFERENCES

1. Apostolopoulos, V., Yuriev, E., Ramsland, P. A., Halton, J., Osinski, C., Li, W., Plebanski, M., Paulsen, H., & Mckenzie, I. F. C. (2003). A glycopeptide in complex with MHC class I uses the GalNAc residue as an anchor. Proceedings of the National Academy of Sciences of the United States of America, 100 (25). doi.org/10.1073/pnas.2432220100
2. Chalabi, M., Fanchi, L. F., Dijkstra, K. K., Van den Berg, J. G., Aalbers, A. G., Sikorska, K., Lopez-Yurda, M., Grootscholten, C., Beets, G. L., Snaebjornsson, P., Maas, M., Mertz, M., Veninga, V., Bounova, G., Broeks, A., Beets-Tan, R. G., de Wijkerslooth, T. R., van Lent, A. U., Marsman, H. A., . . . . Haanen, J. B. (2020). Neoadjuvant immunotherapy leads to pathological responses in MMR-proficient and MMR-deficient early-stage colon cancers. Nature Medicine, 26 (4). doi.org/10.1038/s41591-020-0805-8
3. Davar, D., Dzutsev, A. K., McCulloch, J. A., Rodrigues, R. R., Chauvin, J. M., Morrison, R. M., Deblasio, R. N., Menna, C., Ding, Q., Pagliano, O., Zidi, B., Zhang, S., Badger, J. H., Vetizou, M., Cole, A. M., Fernandes, M. R., Prescott, S., Costa, R. G. F., Balaji, A. K., . . . . Zarour, H. M. (2021). Fecal microbiota transplant overcomes resistance to anti-PD-1 therapy in melanoma patients. Science, 371 (6529). doi.org/10.1126/science.abf3363
4. Gabba, A., Attariya, R., Behren, S., Pett, C., van der Horst, J. C., Yurugi, H., Yu, J., Urschbach, M., Sabin, J., Birrane, G., Schmitt, E., van Vliet, S. J., Besenius, P., Westerlind, U., & Murphy, P. V. (2023). MUC1 Glycopeptide Vaccine Modified with a GalNAc Glycocluster Targets the Macrophage Galactose C-type Lectin on Dendritic Cells to Elicit an Improved Humoral Response. Journal of the American Chemical Society, 145 (24). doi.org/10.1021/jacs.2c12843
5. Gandini, A., Puglisi, S., Pirrone, C., Martelli, V., Catalano, F., Nardin, S., Seeber, A., Puccini, A., & Sciallero, S. (2023). The role of immunotherapy in microsatellites stable metastatic colorectal cancer: state of the art and future perspectives. In Frontiers in Oncology (Vol. 13). doi.org/10.3389/fonc.2023.1161048
6. Gray, M. A., Stanczak, M. A., Mantuano, N. R., Xiao, H., Pijnenborg, J. F. A., Malaker, S. A., Miller, C. L., Weidenbacher, P. A., Tanzo, J. T., Ahn, G., Woods, E. C., Läubli, H., & Bertozzi, C. R. (2020). Targeted glycan degradation potentiates the anticancer immune response in vivo. Nature Chemical Biology, 16 (12). doi.org/10.1038/s41589-020-0622-x
7. Johnson, D. B., Nebhan, C. A., Moslehi, J. J., & Balko, J. M. (2022). Immune-checkpoint inhibitors: long-term implications of toxicity. In Nature Reviews Clinical Oncology (Vol. 19, Issue 4). doi.org/10.1038/s41571-022-00600-w
8. Karlsson, H., Larsson, J. M. H., Thomsson, K. A., Hard, I., Bäckström, M., & Hansson, G. C. (2009). High-throughput and high-sensitivity nano-LC/MS and MS/MS for O-glycan profiling. Methods in Molecular Biology, 534, 117-131. doi.org/10.1007/978-1-59745-022-5_9
9. Lavelle, E. C., & Ward, R. W. (2022a). Mucosal vaccines-fortifying the frontiers. In Nature Reviews Immunology (Vol. 22, Issue 4). doi.org/10.1038/s41577-021-00583-2
10. Lavelle, E. C., & Ward, R. W. (2022b). Publisher Correction: Mucosal vaccines fortifying the frontiers. Nature Reviews Immunology, 22 (4). doi.org/10.1038/s41577-021-00599-8
11. Lin, K. X., Istl, A. C., Quan, D., Skaro, A., Tang, E., & Zheng, X. (2023). PD-1 and PD-L1 inhibitors in cold colorectal cancer: challenges and strategies. In Cancer Immunology, Immunotherapy (Vol. 72, Issue 12). doi.org/10.1007/s00262-023-03520-5
12. Lodewijk, I., Dueñas, M., Paramio, J. M., & Rubio, C. (2023). CD44v6, STn & O-GD2: promising tumor associated antigens paving the way for new targeted cancer therapies. Frontiers in Immunology, 14. doi.org/10.3389/FIMMU.2023.1272681
13. Mayordomo, J., Tres, A., Miles, D., Finke, L., & Jenkins, H. (2004). Long-term follow-up of patients concomitantly treated with hormone therapy in a prospective controlled randomized multicenter clinical study comparing STn-KLH vaccine with KLH control in stage IV breast cancer following first-line chemotherapy. Journal of Clinical Oncology, 22 (14_suppl). doi.org/10.1200/jco.2004.22.90140.2603
14. Parker, R., Partridge, T., Wormald, C., Kawahara, R., Stalls, V., Aggelakopoulou, M., Parker, J., Powell Doherty, R., Ariosa Morejon, Y., Lee, E., Saunders, K., Haynes, B. F., Acharya, P., Thaysen-Andersen, M., Borrow, P., & Ternette, N. (2021). Mapping the SARS-COV-2 spike glycoprotein-derived peptidome presented by HLA class II on dendritic cells. Cell Reports, 35 (8). doi.org/10.1016/j.celrep.2021.109179
15. Pinho, S. S., & Reis, C. A. (2015). Glycosylation in cancer: mechanisms and clinical implications. Nature Reviews Cancer, 15 (9), 540-555. doi.org/10.1038/nrc3982
16. Reily, C., Stewart, T. J., Renfrow, M. B., & Novak, J. (2019). Glycosylation in health and disease. In Nature Reviews Nephrology (Vol. 15, Issue 6). doi.org/10.1038/s41581-019-0129-4
17. Rodríguez, E., Schetters, S. T. T., & van Kooyk, Y. (2018). The tumour glyco-code as a novel immune checkpoint for immunotherapy. Nature Reviews Immunology, 18 (3), 204-211. doi.org/10.1038/nri.2018.3
18. Rosenbaum, P., Artaud, C., Bay, S., Ganneau, C., Campone, M., Delaloge, S., Gourmelon, C., Loirat, D., Medioni, J., Pein, F., Sablin, M. P., Tredan, O., Varga, A., & Leclerc, C. (2020). The fully synthetic glycopeptide MAG-Tn3 therapeutic vaccine induces tumor-specific cytotoxic antibodies in breast cancer patients. Cancer Immunology, Immunotherapy, 69 (5). doi.org/10.1007/s00262-020-02503-0
19. Sandoval, F., Terme, M., Nizard, M., Badoual, C., Bureau, M. F., Freyburger, L., Clement, O., Marcheteau, E., Gey, A., Fraisse, G., Bouguin, C., Merillon, N., Dransart, E., Tran, T., Quintin-Colonna, F., Autret, G., Thiebaud, M., Suleman, M., Riffault, S., . . . . Tartour, E. (2013). Mucosal imprinting of vaccine-induced CD8+ T cells is crucial to inhibit the growth of mucosal tumors. Science Translational Medicine, 5 (172). doi.org/10.1126/scitranslmed.3004888
20. Siegel, R. L., Wagle, N. S., Cercek, A., Smith, R. A., & Jemal, A. (2023). Colorectal cancer statistics, 2023. CA: A Cancer Journal for Clinicians, 73 (3). doi.org/10.3322/caac.21772
21. Sosa Cuevas, E., Roubinet, B., Mouret, S., Thépaut, M., de Fraipont, F., Charles, J., Fieschi, F., Landemarre, L., Chaperot, L., & Aspord, C. (2023). The melanoma tumor glyco-code impacts human dendritic cells' functionality and dictates clinical outcomes. Frontiers in Immunology, 14. doi.org/10.3389/fimmu.2023.1120434
22. Suijkerbuijk, K. P. M., van Eijs, M. J. M., van Wijk, F., & Eggermont, A. M. M. (2024). Clinical and translational attributes of immune-related adverse events. In Nature Cancer. doi.org/10.1038/s43018-024-00730-3
23. Sun, L., Paschall, A. V., Middleton, D. R., Ishihara, M., Ozdilek, A., Wantuch, P. L., Aceil, J., Duke, J. A., LaBranche, C. C., Tiemeyer, M., & Avci, F. Y. (2020). Glycopeptide epitope facilitates HIV-1 envelope specific humoral immune responses by eliciting T cell help. Nature Communications, 11 (1). doi.org/10.1038/s41467-020-16319-0
24. Van Gisbergen, K. P. J. M., Aarnoudse, C. A., Meijer, G. A., Geijtenbeek, T. B. H., & Van Kooyk, Y. (2005). Dendritic cells recognize tumor-specific glycosylation of carcinoembryonic antigen on colorectal cancer cells through dendritic cell-specific intercellular adhesion molecule-3-grabbing nonintegrin. Cancer Research, 65 (13). doi.org/10.1158/0008-5472.CAN-04-4140
25. Varki, A., Cummings, R. D., Esko, J. D., Stanley, P., Hart, G. W., Aebi, M., Darvill, A. G., Kinoshita, T., Packer, N. H., Prestegard, J. H., Schnaar, R. L., & Seeberger, P. H. (2022). Essentials of glycobiology. In Cold Spring Harbor Laboratory Press. worldwideweb.ncbi.nlm.nih.gov/books/NBK579918/

1. A method for treating cancer in a mammal in need thereof wherein a standard therapy for the cancer is 5FU, the method comprising administering to the mammal an effective amount of a composition comprising a plurality of glycopeptides,
wherein at least 90% of the plurality of glycopeptides in the composition are less than 2 KDa in size, and
wherein the plurality of glycopeptides comprises the following oligosaccharide structures:
Fucα1-2Galβ1-3GalNAc, Galβ1-3(GlcNAcβ1-6)GalNAc1, Galβ1-3(6SGlcNAcβ1-6)GalNAc, Galβ1-3(NeuAcα2-6)GalNAc2, NeuAcα2-3Galβ1-3GalNAc3, Galβ1-3(NeuGcα2-6)GalNAc, NeuGcα2-3Galβ1-3GalNAc, GlcNAcβ1-3(6S-GlcNAcβ1-6)GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, NeuAcα2-3Galβ1-3 [(6S)GlcNAcβ1-6]GalNAc, NeuAcα2-3Galβ1-3(NeuAcα2-6)GalNAc, NeuGcα2-3Galβ1-3(NeuAcα2-6)GalNAc, NeuGcα2-3Galβ1-3(NeuGcα2-6)GalNAc, and GalNAcα1-3 (Fucα1-2)Galβ1-3(6SGlcNAcβ1-6)GalNAc;
thereby treating the cancer in the recipient mammal.

2. The method of claim 1, wherein the cancer is selected from melanoma, breast cancer, lung cancer or colorectal cancer (CRC).

3. The method of claim 1, wherein the cancer is Microsatellite stable (MSS) colorectal cancer.

4. The method of claim 1, wherein the composition is administered to the mammal for 28 days or more.

5. The method of claim 1, wherein the plurality of glycopeptides is derived from porcine gastrointestinal mucins, and wherein the composition is obtained without subjecting the porcine gastrointestinal mucins to conditions or reagents that cause release of oligosaccharides from glycopeptides.

6. The method of claim 1, wherein the cancer is stage 3 or 4 cancer.

7. The method of claim 1, wherein the cancer is resistant to checkpoint inhibitor therapy.

8. The method of claim 1, further comprising administering to the recipient mammal an immune checkpoint inhibitor before, after, or simultaneously with the composition.

9. The method of claim 8, the immune checkpoint inhibitor is an anti-PD1 antibody or an anti-PDL1 antibody.

10. A method for preparing reprogrammed T cells comprising:
administering to a donor mammal an effective amount of a composition comprising a plurality of glycopeptides,
wherein at least 90% of the plurality of glycopeptides in the composition are less than 2 KDa in size, and
wherein the plurality of glycopeptides comprises the following oligosaccharide structures:
Fucα1-2Galβ1-3GalNAc, Galβ1-3(GlcNAcβ1-6)GalNAc1, Galβ1-3(6SGlcNAcβ1-6)GalNAc, Galβ1-3(NeuAcα2-6)GalNAc2, NeuAcα2-3Galβ1-3GalNAc3, Galβ1-3(NeuGcα2-6)GalNAc, NeuGcα2-3Galβ1-3GalNAc, GlcNAcβ1-3(6S-GlcNAcβ1-6)GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, NeuAcα2-3Galβ1-3 [(6S)GlcNAcβ1-6]GalNAc, NeuAcα2-3Galβ1-3(NeuAcα2-6)GalNAc, NeuGcα2-3Galβ1-3(NeuAcα2-6)GalNAc, NeuGcα2-3Galβ1-3(NeuGcα2-6)GalNAc, and GalNAcα1-3 (Fucα1-2)Galβ1-3(6SGlcNAcβ1-6)GalNAc; and
isolating the reprogrammed T cells from the donor mammal, wherein the reprogrammed T cells are capable of targeting tumor cells in a recipient mammal.

11. The method of claim 10, further comprising cryopreserving the isolated T cells.

12. The method of claim 10, wherein the composition is administered to the donor mammal for 28 days or more before isolating the T cells.

13. The method of claim 10, wherein the glycopeptides were derived from porcine gastrointestinal mucins, and wherein the composition is obtained without subjecting the porcine gastrointestinal mucins or the partially purified fraction thereof to conditions or reagents that cause complete release of oligosaccharides from glycopeptides.

14. An ex vivo or in vitro method for preparing reprogrammed T cells comprising:
Contacting T cells from a mammal with an effective amount of a composition comprising a plurality of glycopeptides,
wherein at least 90% of the plurality of glycopeptides in the composition are less than 2 KDa in size, and
wherein the plurality of glycopeptides comprises the following oligosaccharide structures:
Fucα1-2Galβ1-3GalNAc, Galβ1-3(GlcNAcβ1-6)GalNAc1, Galβ1-3(6SGlcNAcβ1-6)GalNAc, Galβ1-3(NeuAcα2-6)GalNAc2, NeuAcα2-3Galβ1-3GalNAc3, Galβ1-3(NeuGcα2-6)GalNAc, NeuGcα2-3Galβ1-3GalNAc, GlcNAcβ1-3(6S-GlcNAcβ1-6)GalNAc, Galβ1-3(Galβ1-4GlcNAcβ1-6)GalNAc, Fucα1-2Galβ1-3(6S-GlcNAcβ1-6)GalNAc, Galβ1-3(Galα1-3Galβ1-4GlcNAcβ1-6)GalNAc, NeuAcα2-3Galβ1-3 [(6S)GlcNAcβ1-6]GalNAc, NeuAcα2-3Galβ1-3(NeuAcα2-6)GalNAc, NeuGcα2-3Galβ1-3(NeuAcα2-6)GalNAc, NeuGcα2-3Galβ1-3(NeuGcα2-6)GalNAc, and GalNAcα1-3 (Fucα1-2)Galβ1-3(6SGlcNAcβ1-6)GalNAc; and
isolating the reprogrammed T cells, wherein the reprogrammed T cells are capable of targeting tumor cells in a recipient mammal.

15. The method of claim 14, wherein the reprogrammed T cells are used for adoptive cell transfer.
